(19) United States
(12) Patent Application Publication
(10) Pub. No.: US 2016/0106536 A1
(43) Pub. Date: Apr. 21, 2016

(54) Methods and Materials for an Artificial Voice Prosthesis

(71) Applicants: NATIONAL UNIVERSITY OF SINGAPORE, Singapore (SG); SINGAPORE HEALTH SERVICES PTE LTD, Singapore (SG)

(72) Inventors: Chee Kong Chui, Singapore (SG); Chin Heng Matthew Chua, Singapore (SG); Pang Cheng David Lau, Singapore (SG); Ee Hoon Constance Teo, Singapore (SG)

(21) Appl. No.: 14/894,743

(22) PCT Filed: May 30, 2014
(86) PCT No.: PCT/SG2014/000251

§ 371 Date: Nov. 30, 2015

Related U.S. Application Data
(60) Provisional application No. 61/829,370, filed on May 31, 2013.

(51) Int. Cl. A61F 2/20; A61L 27/44

(52) U.S. Cl. CPC A61F 2/203; A61L 27/443; A61F 2240/002

(57) ABSTRACT

A voice prosthesis includes a body carrying a passage, and a magnetic passage sealing mechanism having a ball that can selectively seal/block or open the passage. The voice prosthesis can include an outer skin that covers the body. The voice prosthesis can include a polymer carrying a nanomaterial. The voice prosthesis can be fabricated as a patient specific device in accordance with images of a fistula of a target patient for whom the voice prosthesis is intended.

### TECHNICAL FIELD

Aspects of the present disclosure relate to a methodology, material and system for the design, fabrication and deployment of a patient specific artificial tracheal prosthesis and/or a patient specific voice prosthesis.

### BACKGROUND

There is a need in the medical and healthcare industry to create suitable or optimized implantable medical devices, including organ replacement devices, which can be patient specific organ replacement devices as there may be significant differences in the anatomical dimensions from individual to individual. To date, there has not been a commercially successful tracheal prosthesis product due to several limitations faced in the animal experimental and clinical stage.

High failure rates of the tracheal prosthesis are attributed to the lack of mechanical strength to withstand surrounding pressure, lack of flexibility compared to natural tracheal tissues, slow rate of growth of ciliated epitheliazation and leakage of interstitial fluid into the lumen.

### Limitations of Existing Tracheal Prostheses

The limitation of existing tracheal prosthesis is the high rate of failure due to stenosis or narrowing of the lumen after implantation. This could be due to inadequate mechanical strength to withstand external pressure thus resulting in the collapse of the lumen. Another reason could be inadequate epitheliazation into the implant which causes granulation tissues to form and failure to occur. The issue of leakage of interstitial fluid from the surrounding into the trachea due to poor sealing can result in device failure at the proximal ends due to anastomotic tension. Implant porosity is another factor that can potentially cause fatal pneumonia due to fluid leakage. Also, cell seeding of the patient's own tracheal ciliated cells onto a decellularized tracheal scaffold in a bioreactor is time consuming and very costly, which may not be suitable for emergency cases.

### Limitations of Existing Voice Prostheses

For a voice prosthesis, several companies like Provox™ have a wide range of voice prosthesis products in the market. Existing voice prostheses have several limitations, one of which is that they come in fixed range of sizes and a universal shape. This may result in periprosthetic leakage due to misfit of the prosthesis in the fistula due to the actual shape of the fistula. This may also arise when the fistula widens over time. Also, transprosthetic leakage due to the incomplete closure of the valve could accelerate Candida formation which greatly reduces the lifespan of the prosthesis. All of these cause much inconvenience to the patient as they will be required to clean their prosthesis more frequently, as well as spend more money to replace the device within a shorter period of time than expected.

A need clearly exists for improved tracheal and voice prostheses, which would overcome at least some of the aforementioned limitations.

### SUMMARY

Embodiments in accordance with the present disclosure overcome at least some of the above-mentioned limitations by way of a system and proposed methodology of computer simulation, design and fabrication of an artificial tracheal prosthesis and/or an artificial voice prosthesis for implantation. In some embodiments, at least a portion of the tracheal prosthesis and/or at least a portion of the voice prosthesis can have a patient specific design or geometry. For the artificial trachea, a finite element analysis simulation can be based on a virtual tracheal model reconstructed from a set of volume images or volumetric images obtained from an imaging scan of a target patient/subject, such as a CT/MRI scan of the target patient. This allows for a realistic simulation environment to test the suitability of a designed prosthesis. A patient specific artificial trachea can then be fabricated using a rapid prototyping or additive manufacturing technique such as 3D-printing with one or more types of materials, such as at least one type of biocompatible polymer material that carries at least one type of nanomaterial. A tracheal prosthesis in accordance with an embodiment of the present disclosure can thus include a carbon nanocomposite material that has similar mechanical properties to the native tissue. In a representative embodiment, a tubular or generally tubular tracheal prosthesis having an outer surface, an inner surface, a first end, a second end, a length between the first and second ends, and an inner lumen extending along the inner surface between the first and second ends can be fabricated or formed as a material matrix that includes polydimethylsiloxane (PDMS) and at least one nanomaterial, such as single wall carbon nanotubes (SWCNTs), multiwalled carbon nanotubes (MWCNT), carbon nanofibers, nanospheres, one or more other carbon-based nanomaterials, and/or one or more non-carbon based nanomaterials. For purpose of simplicity and to aid understanding, representative embodiments considered herein include SWCNTs. Manufacturing using bioprinters or multi-material electrohydrodynamic jet printers can also be used for fabrication. The methodologies in accordance with embodiments of the present disclosure, and/or similar, methodologies, can also be applied to other tubular or generally tubular tissue replacements such as replacement of portions of vascular vessels, nerves and/or intestines.

For a voice prosthesis in accordance with an embodiment of the present disclosure, volumetric imaging of the patient's fistula or an image of the geometrical area of a target patient's fistula as well as the corresponding depth is captured. The image is then processed via image processing software and a voice prosthesis model with a patient specific shape is reconstructed. Allowances for dimensions are made to ensure a snug fit. Afterwards, a mold can be rapid-prototyped out to facilitate or enable the manufacturing of a patient-specific voice prosthesis, such as a nanotube-polymer composite voice prosthesis. Alternatively, the prosthesis can be rapid-prototyped out and subjected to in-vitro and/or in-vivo testing. A multi-layered voice prosthesis skin that has an inner soft PVC sponge or silicone gel and a CNT-PDMS nanocomposite outer layer or covering can also be formed in accordance with an embodiment of the present disclosure. The multi-layered skin allows for the adaptations of the prosthesis to small changes in the patient's fistula.

### Representative Methodology for Artificial Trachea

In one aspect, an embodiment in accordance with the present disclosure provides a methodology for the evaluation of the performance quality of a designed tracheal prosthesis in a simulated patient specific tracheal environment. This initial stage of design is based on volume images captured from the patient's trachea via clinical devices such as x-ray, magnetic resonance imaging (MRI), ultrasound techniques (US), computerized tomography (CT) scanners, rotational angiography and/or other imaging modalities. Preferably, the patient being scanned should be the one suffering from a tracheal disorder or disease like stenosis or cancer, and requires treatment. This methodology enables replication of the characteristics of a diseased region of tracheal tissue for more realistic simulation of the deployment and performance evaluation of the designed prosthesis.

From the collated volume images of the patient, the geometry of the diseased or dysfunctional portion(s) of the trachea to be excised can be used to determine the dimensions of the prosthesis. In one aspect, the volume images of the trachea will be processed and segmented, following which, a 3D model of the patient's trachea is reconstructed in a computer-aided design (CAD) software like Solidworks™. As the trachea consists primarily of 2 different tissues, namely the membranous tissues and the cartilage rings, each with their own different biomechanical properties and behaviors, such parameters relating to their properties based on known information can be input into their respective regions for simulation purposes.

In one aspect, an embodiment in accordance with the present disclosure also provides a methodology for the designing and evaluation of a tracheal prosthesis. Based on the initial collected volume images of the patient's trachea, the radii of the minor and major axes of the target patient's elliptic natural tracheal rings are used to determine the dimensions of the inner non-biodegradable ellipse-shaped prosthesis scaffold. The thickness of the membranous tissue surrounding the cartilage rings can be used as a gauge to determine the thickness of a collagen sponge matrix required to cover the non-biodegradable scaffold. The final artificial trachea prosthesis is a multi-material device, which includes an inner non-biodegradable scaffold that is typically made of a composite material including carbon nanotubes as fibers and poly-di-methyl-siloxane (PDMS) as the matrix, due to its similarity in mechanical properties to the native tracheal rings and its biocompatibility. It will also be structured in an ellipse shape that is similar in dimension to the patient's tracheal rings. The proximal ends of the tubular prosthesis are wrapped with a layer of Dacron to make it leak proof Next, the nanotubes-PDMS composite skeleton is coated with a thick layer of solidified Type I collagen sponge on both the inner and outer sides. This biodegradable layer serves as a temporary matrix for the in growth of cells and blood vessels into the prosthesis. Furthermore, since collagen is essentially a majority part of the surrounding membranous tissue, it will be easily integrated. The collagen matrix is also loaded with at least one growth factor, such as the protein Vascular Endothelial Growth Factor (VEGF), just before implantation, which helps to stimulate and accelerate the ingrowth of blood vessels and cells into the prosthesis. The VEGF can be encapsulated in a Poly Electrolyte Complex (PEC) to prolong its lifespan in vivo.

In another embodiment in accordance with the present disclosure, it provides a methodology for the design of the patient specific tracheal prosthesis. Based on the initial collected volumetric images of the patient's trachea, the dimensions of the minor and major axis of the elliptic cartilage rings are used to determine the dimensions of the skeletal backbone or scaffold of the prosthesis that will provide mechanical strength while maintaining airway patency.

The thickness of the natural trachea wall (e.g., which can be a typical or average tracheal wall thickness across multiple patients, or an estimated/measured patient specific tracheal wall thickness based upon a set of anatomical images of a target patent under consideration) can be used to determine the thickness of a collagen matrix or sponge layer carried by or coating the inner lumen of the skeletal scaffold. In various embodiments, the collagen matrix/sponge layer extends only partially, and not fully, along or through the entire scaffold. More particularly, in some embodiments, at and proximate to each of the first end and the second end of the scaffold, the inner surface of the lumen is exposed, and not covered or overlaid with the collagen matrix/sponge layer. Rather, the collagen matrix/sponge layer overlays or covers the inner surface of the lumen beyond a certain distance away from each of the scaffold's first and second ends into the depth of the scaffold's lumen. For instance, in a representative embodiment, for a scaffold having a length L or a depth D, the collagen matrix/sponge layer can occupy approximately 90%-95% of the inner surface of the lumen along the scaffold's length L or depth D, and the collagen matrix/sponge layer can be absent from the inner surface of the lumen along approximately 5%-10% of the inner surface of the lumen corresponding to the scaffold's first and second ends. The presence of the collagen matrix/sponge layer on the inner surface of the lumen, and the absence of the collagen matrix/sponge layer at and proximate to the scaffold's first and second ends, results in the formation of a step structure within the lumen of the prosthesis to facilitate or enable the creation of a flush surface between the top surface of the collagen matrix/sponge layer and the patient's natural ciliated epithelium when the resected ends of the natural trachea are inserted partially into the depth of the lumen for anastomosis. The final artificial tracheal prosthesis is a multi-material device. Its non-biodegradable skeletal scaffold is preferably made of a biocompatible composite material consisting of carbon nanotubes and polydimethylsiloxane (PDMS). This allows for tailoring of its mechanical properties to be similar to that of the native tracheal rings. The layer of biodegradable collagen sponge coating the lumen serves as a temporary matrix for the in growth of cells and blood vessels into the prosthesis. Furthermore, the flushed collagen matrix/sponge layer will guide the migration of the ciliated epithelium from the ends of the prosthesis into or along the depth or length of the prosthesis. This reduces the chances of implant failure and aids in mucus removal. The collagen matrix can also be loaded with at least one type of growth factor, such as the protein Vascular Endothelial Growth Factor (VEGF) and/or human Epithelial Growth Factor (hEGF) just before implantation, which helps to stimulate and accelerate the ingrowth of blood vessels and cells into the prosthesis.

In one aspect, an embodiment in accordance with the present disclosure also provides a methodology for the integration of the tracheal prosthesis in the constructed patient specific tracheal model for simulation and performance evaluation. In addition to CNT-PDMS nanocomposite, 316L stainless steel was also tested as a scaffold material for simulation and comparison, due to its strength and ability to withstand compressive forces under tracheal loading conditions, as well as its biocompatibility in the host body. Using Solidworks™ assembly module, two cartilage rings from the diseased portion (central in this case) were removed and replaced with the designed scaffold. This is to simulate the real life scenario, whereby the entire collagen matrix has been broken down and only the scaffold is left embedded in the newly formed membranous tissues. For comparison purposes in the simulation, both a modular circular shaped hollow prosthesis and a patient specific prosthesis were designed using Solidworks™. Material properties of both 316L stainless steel and PDMS bio-composite were also input into both geometrical designs. The assembly file was then put through stretching and bending motions in COMSOL™ Multi-physics to study the stress concentrations in the different regions during daily motions of the trachea. The results show that CNT-PDMS bio-composite or CNT-PDMS nanocomposite is a suitable or desirable material of choice (e.g., compared to 316L stainless steel) for the scaffold due to closer similarities in stress concentrations to the native tracheal rings and membrane. Similarly, results data also pointed out the reduction in stress concentrations when a user centric design is used rather than a modular geometrical shape.

In one aspect, an embodiment in accordance with the present disclosure also provides a fabrication methodology for quicker and timely production of a tracheal implant device. As tracheal replacement surgeries may be potentially urgent, the duration, availability and ease of fabrication is paramount to the survivability of the patient. In one embodiment of the present disclosure, the methodology employs the use of rapid prototyping (RP) to create a mold for the curing of PDMS bio-composite due to its intricate geometry and shape. Once a cured scaffold is obtained, another mold, which can be made of aluminium or another suitable material, is machined out for the heating and dry freezing of the collagen matrix together with the PDMS composite. A certain predetermined time such as a predetermined number of hours, for instance, 12 hours, is a suitable or optimal duration of an oven baking process of the collagen matrix in order to yield the longest degradation time. In another embodiment of the present disclosure, the methodology employs the use of rapid prototyping (RP) or 3D printing to fabricate the carbon nanocomposite due to its intricate geometry and the speed of RP. 3D printing allows for filaments of different materials to be printed easily. Carbon nanocomposite filaments of different CNT compositions were fabricated beforehand and preloaded into the 3D printer according to a set of desired mechanical properties. A multi-material electrohydrodynamic jet printer can also be used for the fabrication of the carbon nanocomposite device, as it can have good or better control over the spatial composition of the implant. Another mold was manufactured for the heating and dry freezing of the collagen sponge. A certain predetermined time such as a predetermined number of hours, for instance, 12 hours, is a suitable or optimal duration of the oven baking process of the collagen matrix. The collagen sponge and the nanocomposite skeleton are then joined together using bioglue Coseal™.

Additionally, an embodiment in accordance with the present disclosure also includes a deployment process. In this portion, the diseased portion of the trachea is first removed. In one embodiment of the present disclosure, the tracheal prosthesis is then soaked in pre-prepared solution of VEGF (Vascular endothelial growth factor) encapsulated in poly electrolyte complex (PEC) to improve the life span of the growth factor in vivo. Following which, the prosthesis is soaked in the patient's own blood medium to render it air tight, before connecting and suturing it to the trachea. In another embodiment of the present disclosure, the tracheal prosthesis is soaked in pre-prepared solution of VEGF and hEGF. Following which, the prosthesis is soaked in the patient's own blood medium to render it air tight. The resected ends of the trachea are inserted into the lumen of the prosthesis until it is in contact with the collagen sponge and the lumen surfaces are flush with one another. The CNT-PDMS skeleton thus acts as a sheath to the resected ends of native trachea. The prosthesis and trachea are then sutured together using 3-0 biodegradation Polysorb™ sutures.

Aspects of Tracheal Prostheses in Accordance with the Present Disclosure

In accordance with the present disclosure, representative embodiments of particular processes 20a, 20b for providing, forming, or fabricating a tracheal prosthesis 100 are shown in FIGS. 1A and 1B, where the tracheal prosthesis 100 can have a structure such as that illustrated in FIGS. 4A, 4B, and 14.

More particularly, in accordance with an aspect of the present disclosure, a tracheal prosthesis 100 includes a scaffold 110 formed as a generally tubular structure, the scaffold having an outer surface 112, a first end 114, a second end 116, a length between the first end 114 and the second end 116, and a lumen 120 extending between the first end and the second end and having an inner surface 122. The lumen 120 can have an elliptical cross section. The scaffold 110 includes a material matrix 130 formed of at least one biocompatible polymer carrying at least one nanomaterial. The at least one biocompatible material can include or be polydimethylsiloxane (PDMS), and the at least one nanomaterial can include or be carbon nanotubes (CNT). The scaffold 110 can include a plurality of apertures 135 radially disposed along portions of its length. The scaffold 110 can formed by way of rapid prototyping/additive manufacturing, and/or molding.

The scaffold 110 further carries a collagen matrix layer 140 disposed at least along portions of the inner surface 122 of the lumen 120 of the scaffold 110. The collagen matrix layer 140 can carry at least one growth factor. The growth factor can include or be at least one of vascular endothelial growth factor (VEGF) and epithelial growth factor (EGF). At and proximate to the first end 114 and the second end 116 of the scaffold 110 within the lumen 120, the collagen matrix layer 140 can be absent from the inner surface 122 of the lumen 120. The presence of the collagen matrix layer 140 on the inner surface 122 of the lumen 120, and the absence of the collagen matrix layer 140 on the inner surface 122 of the lumen 120 at and proximate to the first and second ends 114, 116 of the scaffold 110 forms a step structure 142 within the lumen. The step structure 142 facilitates creation of a flush surface 144 between a top surface of the collagen matrix layer 140 and ciliated epithelium tissue when resected ends of a patient's natural trachea are inserted partially into the depth of the lumen 120.

Depending upon embodiment details or a clinical situation under consideration, the tracheal prosthesis 100 can have a non patient specific geometry, or a patient specific geometry. For instance, at least a portion of the scaffold 110 can have a patient specific geometry that matches a tracheal geometry of a target patient based upon a set of anatomical images of the target patient's trachea. A thickness of the collagen matrix layer 140 can be determined based upon a thickness of the target patient's natural tracheal wall as determined by way of an analysis of the set of anatomical images. Elliptic dimensions of the lumen 120 can correspond to radii of minor and major axes of the target patient's elliptic natural tracheal rings as determined from the set of anatomical images. The scaffold's patient specific geometry can be intended to structurally compensate for a diseased or dysfunctional region of the target patient's natural trachea.

In accordance with an aspect of the present disclosure, a process 20a, 20b for producing a tracheal prosthesis 100 includes providing a scaffold 110 as a generally tubular structure having a material matrix 130 formed of at least one biocompatible polymer carrying at least one nanomaterial, the scaffold having an outer surface 112, a first end 114, a second end 116, a length between the first end 114 and the second end 116, and a lumen 120 (e.g., having an elliptical cross section) having an inner surface 122 and extending between the first end 114 and the second end 116 of the scaffold 110; and disposing a collagen matrix layer 140 on portions of the inner surface 122 of the lumen 120, wherein at and proximate to the first end 114 and the second end 116 of the scaffold 110 the lumen 120, the collagen matrix layer 140 is absent from the inner surface 122 of the lumen 120, such that the presence of the collagen matrix layer 140 on portions of the inner surface 122 of the lumen 120 and the absence of the collagen matrix layer 140 on the inner surface 122 of the lumen 120 at and proximate to the first and second ends 114, 116 of the scaffold 110 forms a step structure 142 within the lumen. The step structure 142 can facilitate the provision of a flush surface 144 between a top surface of the collagen matrix layer 140 and ciliated epithelium tissue when resected ends of a patient's natural trachea are inserted partially into the depth of the lumen 120.

The process 20a, 20b can further include capturing a set of anatomical images of a trachea of a target patient; and analyzing the set of captured anatomical images to determine a set of geometric or dimensional parameters of the target patient's trachea. Providing the scaffold 110 can thus include fabricating the scaffold 110 such that the lumen 120 of the scaffold has a geometry or dimensions determined in accordance with the set of geometric or dimensional parameters determined for the target patient's trachea. Analyzing the set of captured images to determine the set of geometric or dimensional parameters can include: determining radii of minor and major axes of the target patient's elliptic natural tracheal rings using the set of captured anatomical images; and incorporating the determined radii into the set of geometric or dimensional parameters. The set of captured anatomical images can include a diseased or dysfunctional region of the target patient's trachea. The set of geometric or dimensional parameters can include parameters intended to structurally compensate for the diseased or dysfunctional region of the patient's trachea.

The process 20b can further include analyzing the set of captured anatomical images to determine a thickness of the target patient's natural tracheal wall. Disposing the collagen matrix layer 140 on portions of the inner surface 122 of the lumen 120 can include disposing the collagen matrix layer 140 to have a thickness expected to approximately match the determined thickness of the target patient's natural tracheal wall

The process 20a, 20b can further include generating a 3D virtual scaffold model that numerically represents the scaffold 110 in accordance with the set of geometric or dimensional parameters. Providing the scaffold 110 can thus include fabricating the scaffold 110 in accordance with the 3D virtual scaffold model by way of one of rapid prototyping/additive manufacturing and/or a mold.

The process 20a, 20b can further include simulating performance characteristics of the scaffold 110 under expected implant conditions by computationally processing the 3D virtual scaffold model to generate at least one of expected stretching and expected bending characteristics of the scaffold 110.

### Representative Methodology for Voice Prosthesis

In one aspect, an embodiment in accordance with the present disclosure provides a methodology for the evaluation and sizing of a fistula for the designing and manufacturing of a patient specific voice prosthesis. The cross section shape and size of the fistula is first captured via a camera or imaging device. The depth of the fistula is also obtained via measurement. Following which, image processing software is used to extract the exact, nearly exact, or approximately exact cross sectional shape and size of the fistula from the captured image and transfer it to CAD software for the designing of a mold of the patient specific voice prosthesis. Dimensional tolerances are factored in to ensure a snug fit of the prosthesis in the fistula.

Another embodiment of this disclosure provides a methodology for designing and manufacturing a patient specific voice prosthesis based on the geometrical dimensions of the fistula. The volumetric shape of the fistula is first captured via clinical imaging device. The volumetric images are then used to reconstruct the patient specific skin model for the voice prosthesis and the mold for its production. Dimensional tolerances are factored in to ensure a snug fit of the prosthesis in the fistula.

In one aspect, an embodiment in accordance with the present disclosure also provides the design of a multi-component voice prosthesis that can easily, switch its outer skin geometrical shape and size according to the patient's fistula. It includes a rigid PVC core to maintain airway patency under compression stresses from surrounding tissues. An adaptable nanocomposite sleeve, which can be or is patient specific and adapts to minor fistula changes, is worn over the rigid core.

In one aspect, an embodiment in accordance with the present disclosure also includes the incorporation of an adaptable and patient specific nanocomposite sleeve. The sleeve is made out of an inner viscous or soft material such as silicone gel or PVC sponge and surrounded by an outer airtight and waterproof layer of CNT-PDMS nanocomposite. The inner adaptable material allows for adaptations of the sleeve to small changes in the dimensions and shape of the patient's fistula. The carbon nanocomposite component, however, has mechanical properties that are tailored to the surrounding tissues and as such reduce stresses and inflammation. The patient specific shape also results in even stress distribution in the surrounding tissue.

In one aspect, an embodiment in accordance with the present disclosure also provides a design methodology of a novel magnetic ball bearing valve or one way valve to prevent transprosthesis leakages as well as slow down the growth of candida, hence prolonging the lifespan of the prosthesis. The magnetic ball bearing valve also has lesser airflow resistance compared to other prostheses. The ball bearing can also vary pitch in the patient's speech and produce a more natural speaking tone.

In one aspect, an evaluation algorithm is proposed in accordance with an embodiment of the present disclosure to ensure comfortable fitting of the voice prosthesis in the patient's fistula as well as ease of opening and closing of the one way valve for speaking.

In one aspect, an embodiment in accordance with the present disclosure also includes the use of bio-composite materials made of carbon nanotubes and PDMS polymer to obtain a strong, yet flexible, voice prosthesis that can conform to the changes in shape of the patient's fistula over time.

Aspects of Voice Prostheses in Accordance with the Present Disclosure

In accordance with the present disclosure, representative embodiments of particular processes 200a, 200b for providing, forming, or fabricating a voice prosthesis 300 are shown in FIGS. 22A and 22B, where the voice prosthesis 300 can have a structure such as that illustrated in FIGS. 23-29.

In accordance with an aspect of the present disclosure, a voice prosthesis 300 includes a body 310 having a length along a body axis and formed of at least one biocompatible polymer; a first surface 320 coupled to the body transverse or perpendicular to the body axis, the first surface 320 having a first aperture 322 disposed therein, the first surface 320 defining a first end 324 of the voice prosthesis 100; a second surface 330 coupled to the body 310 transverse or perpendicular to the body axis, the second surface 330 having a second aperture 332 disposed therein, the second surface 330 defining a second end 334 of the voice prosthesis 300; a passage 340 disposed within the body 310 along at least a portion of the body length between the first end 324 and the second end 334 of the voice prosthesis 300, the passage 340 fluidically coupled to the first aperture 322 and the second aperture 332; and a magnetic sealing mechanism 350 carryable by the body 310 and configured for selectively (a) sealing the passage 340 to prevent airflow through the passage 340 in a direction toward the first aperture 322 in the absence of sufficient air pressure at the first aperture 322, and (b) opening the passage 340 to enable airflow through the passage 340 in a direction toward the second aperture 332 in the presence of sufficient air pressure at the first aperture 322, the magnetic sealing mechanism 350 comprising a ball 352.

The magnetic sealing mechanism 350 can include a retaining link 356 coupled to each of the ball 352 and an inner surface of the passage 340; and a magnetic seating structure 358 carried by the second aperture 332, wherein the magnetic seating structure 358 is configured to shape match a portion of an exterior surface of the ball 352.

The magnetic sealing mechanism 350 can include a magnet 355 or a magnetic material 355m disposed around and/or proximate to the first aperture 322.

The body 310 can be formed to include one of polydimethylsiloxane (PDMS) and polyvinyl chloride (PVC). The body can be formed to include PDMS and at least one nanomaterial, which can include or be carbon nanotubes (CNTs).

The body 310 can include a core structure 312 having an exterior surface, and which carries a chamber 314 in which the ball resides, wherein the chamber 314 is fluidically coupled to the passage 340 and the second aperture 332. A skin layer 360 can be disposed around the exterior surface of the core structure 312, wherein the skin layer 360 forms at least a portion of the second surface 330 of the voice prosthesis 300. The skin layer 360 can include at least one biocompatible polymer carrying at least one nanomaterial, such as PDMS carrying CNTs. The skin layer 360 can include at least one cavity 362 formed therein, in which a deformable material can be disposed.

Depending upon embodiment details or a clinical situation under consideration, the voice prosthesis 300 can have a non patient specific shape, or a patient specific shape. For instance, the body 310 can have a patient specific shape determined in accordance with a set of images of a fistula of a target patient; and/or the skin layer 360 can have a patient specific shape determined in accordance with the set of images of a fistula of the target patient. The set of images includes at least one image generated by way of computed tomography (CT) and a magnetic resonance imaging (MRI).

In accordance with an aspect of the present disclosure, a process 200a,b for producing a voice prosthesis 300 includes providing a body 310 having a length along a body axis and a passage 340 disposed along at least a portion of the body length; providing a first surface 320 coupled to the body 310 transverse or perpendicular to the body axis, the first surface 320 having a first aperture 322 disposed therein, the first surface 320 defining a first end 324 of the voice prosthesis 300; providing a second surface 330 coupled to the body 310 transverse or perpendicular to the body axis, the second surface 330 having a second aperture 332 disposed therein, the second surface 330 defining a second end 334 of the voice prosthesis 300, wherein the passage 340 is fluidically coupled to the first aperture 322 and the second aperture 332; and interfacing a magnetic sealing mechanism 350 with the body 310, the magnetic sealing mechanism 350 comprising a ball 352 configured for selectively (a) sealing the passage 340 to prevent airflow through the passage 340 in a direction toward the first aperture 322 in the absence of sufficient air pressure at the first aperture 332, and (b) opening the passage 340 to enable airflow through the passage 340 in a direction toward the second aperture 332 in the presence of sufficient air pressure at the first aperture 322.

The process 200a,b can include capturing a set of images of a fistula of a target patient; and analyzing the set of captured images to determine a set of fistula parameters that define a fistula shape. Providing the body 310 can thus include forming the body 310 to have an exterior surface that exhibits a geometry or shape determined in accordance with the determined fistula shape. Alternatively, providing the body 310 can include providing a core structure 312 carrying the passage 340 and having a chamber 314 configured for carrying the ball 352, wherein the chamber 314 is fluidically coupled to the passage 340 and the second aperture 332, and the process 200b can further include providing a skin layer 360 configured for covering the core structure 312, wherein when the skin layer 360 covers the core structure 312, the skin layer 360 has an exterior surface that exhibits a shape determined in accordance with the determined fistula shape. When the skin layer 360 covers the core structure 312, a portion of the skin layer 360 can form at least a portion of the second surface 330 of the voice prosthesis 300. Providing the skin layer 360 can include forming the skin layer 360 to include a cavity 362 therein in which a deformable material is disposable. The skin layer 360 can be formed by way of rapid prototyping/additive manufacturing.

The process 200a,b can further include generating a 3D virtual voice prosthesis model that numerically represents the voice prosthesis in accordance with the set of fistula parameters; and simulating performance of the voice prosthesis 300 by computationally processing the 3D virtual voice prosthesis model to generate at least one of voice prosthesis stress characteristics and voice prosthesis airflow characteristics.

### BRIEF DESCRIPTION OF THE DRAWINGS

FIG. 1A is a schematic diagram showing the overall architecture of the system in the design methodology of the artificial trachea in accordance with an embodiment of the present disclosure. Volume images acquired from CT are used to construct a patient specific trachea model, which is then used for the design of the artificial prosthesis and simulation studies.

FIG. 1B is an iterative flow process for the design, simulation, fabrication and experiment of the patient specific artificial trachea in accordance with an embodiment of the present disclosure.

FIG. 2 shows the 3D reconstruction of the (Right) patient specific artificial trachea model using (Left) volumetric images of the scanned trachea.

FIG. 3A shows how the elliptical shape of the prosthesis is determined from the dimensions of the patient's tracheal rings, which are also elliptic in accordance with an embodiment of the present disclosure.

FIG. 3B shows how the cartilage rings in reality have irregular geometry and can be identified from the CT images and imported over to design the prosthesis in accordance with an embodiment of the present disclosure.

FIG. 4A illustrates portions of a scaffold or skeleton of a tracheal prosthesis, or a 3D CAD model corresponding thereo, in accordance with an embodiment of the present disclosure.

FIG. 4B illustrates portions of a scaffold or skeleton of a tracheal prosthesis, or a 3D CAD model corresponding thereto, which can include an inner collagen matrix/sponge layer in accordance with an embodiment of the present disclosure.

FIG. 5 shows how two center rings of a natural trachea are removed and replaced with the prosthesis for Finite Element Analysis (FEA) studies.

FIG. 6 is a schematic diagram showing stress results of the prosthesis, membrane and cartilage rings being measured from the simulated daily bending and stretching motion of the implanted trachea.

FIG. 7A shows the stress results of different implants under different simulated tracheal motion in accordance with an embodiment of the present disclosure. The stress values of the PDMS bio-composite user centric implant is almost similar to the tracheal rings in the natural unmodified trachea model.

FIG. 7B shows the stress results of different implants under different simulated conditions in accordance with an embodiment of the present disclosure. The stress values of the user centric CNT-PDMS implant are almost similar to the tracheal rings in the natural trachea.

FIG. 8A shows the membrane stress results of different implants under different simulated tracheal motion in accordance with an embodiment of the present disclosure. The stress values of the membrane with the implanted user centric PDMS bio-composite prosthesis are almost similar to the membrane in the natural unmodified trachea model.

FIG. 8B shows the membrane stress results for different implants under different simulated conditions in accordance with an embodiment of the present disclosure. The stress values of the membrane with the implanted user centric CNT-PDMS prosthesis are almost similar to the membrane in the natural trachea.

FIG. 9A shows the cartilage rings stress results of different implants under different simulated tracheal motion in accordance with an embodiment of the present disclosure. The stress values of the cartilage rings with the implanted user centric PDMS composite prosthesis are almost similar to the cartilage rings in the natural unmodified trachea model.

FIG. 9B shows the cartilage rings stress results for different implants under different simulated conditions in accordance with an embodiment of the present disclosure. The stress values of the cartilage rings with the implanted user centric CNT-PDMS prosthesis are almost similar to the cartilage rings in the natural trachea.

FIG. 10 shows the composition of the final fabricated tracheal prosthesis in accordance with an embodiment of the present disclosure. It includes a non-biodegradable PDMS-carbon nanotubes composite skeleton wrapped with Dacron at the proximal end and covered with Type I collage sponge matrix that has been loaded with PEC-encapsulated VEGF.

FIG. 11 shows the rapid prototyped mold for the fabrication of the PDMS composite skeleton in accordance with an embodiment of the present disclosure. It includes a twin separable base and a removable top cover to facilitated easy removable of molded part.

FIG. 12 shows the mold for placing the cured PDMS composite skeleton in and for the pouring of the collagen solution to create the collagen sponge matrix in accordance with an embodiment of the present disclosure.

FIG. 13 shows the cross section of a modular circular tubular prosthesis (left) and an elliptical user centric tubular prosthesis (right) in accordance with embodiments of the present disclosure.

FIG. 14 shows the (Left) tapered step design whereby the layer of collagen sponge forms a step on the surface of the CNT-PDMS skeleton and (Right) the suturing of the resected ends of the trachea to the prosthesis with the two surfaces being flushed in accordance with an embodiment of the present disclosure. This helps promote epithelium migration into the prosthesis.

FIG. 15 shows the photo of the final product of the patient specific carbon nanocomposite tracheal prosthesis in accordance with an embodiment of the present disclosure.

FIG. 16 shows the graph of DNA concentration of cells gown on the scaffold in-vitro increasing over a span of one week.

FIG. 17 shows the confocal images of the cells grown on the scaffold in-vitro on (Left) day two and (Right) day 3. Green depicts viable cells while red represents dead cells. There is an increase in viable cell numbers and cell to cell adhesions formed.

FIG. 18 shows the SEM images of the trachea cells grown on the scaffold in-vitro at (A) Day 2 and (B) Day 5. More cilias were observed on Day 5, thus showing the differentiation and cilio-genesis of the trachea cells cultured on the scaffold.

FIG. 19 shows the CT scan of the porcine model (Left) before and (Right) after in-vivo trachea replacement surgery. Blue arrow indicates the implanted prosthesis while the green arrow shows the tube used to maintain anesthesia.

FIG. 20 shows the lumen of the replaced porcine trachea being epithelialized completely by the 2nd week.

FIG. 21 shows the image of the harvested tracheal prosthesis after 3 weeks. The yellow arrow indicates that tissue in-growth has successfully occurred into the prosthesis.

FIG. 22A depicts an overall design methodology of a voice prosthesis in accordance with an embodiment of the present disclosure. Photo of the fistula captured using a camera is processed and used to construct a patient specific voice prosthesis in a CAD software.

FIG. 22B presents an iterative flow process for the design, simulation, fabrication and experiment of a patient specific artificial voice prosthesis in accordance with an embodiment of the present disclosure.

FIG. 23 shows a reconstruction of the patient specific geometry of the voice prosthesis based on the collated volumetric images of the patient's fistula in accordance with embodiments of the present disclosure.

FIG. 24 shows portions of a voice prosthesis or a CAD model corresponding thereto in accordance with an embodiment of the disclosure, in an isometric position with a ball bearing valve closed (left) and the ball bearing separated and showing a magnetic ocular seating (right) structure.

FIG. 25 shows a cross sectional view of the voice prosthesis in close position (left) and in open position while speaking (right) in accordance with an embodiment of the present disclosure.

FIG. 26A shows the cross section of a voice prosthesis in accordance with an embodiment of the present disclosure. It includes an inner rigid PVC core and an interchangeable carbon nanocomposite outer skin that is patient specific. It operates via a magnetic ball bearing valve shown in the open (left) and closed (right) positions.

FIG. 26B shows a cross section of the patient specific carbon nanocomposite voice prosthesis in accordance with an embodiment of the present disclosure.

FIG. 27 presents a 3D isometric model of a (A) hollow CNT-PDMS skin layer and its (B) cross section in accordance with an embodiment of the present disclosure. A cavity in the skin layer allows for the insertion of PVC sponge or gel which can deform and self-adjust for minor changes in the geometry of the patient's fistula.

FIG. 28 presents a 3D model of the (A) reconstructed patient's fistula, (B) regular cylindrical shaped prosthesis and (C) patient specific voice prosthesis in accordance with an embodiment of the present disclosure.

FIG. 29 shows a lateral cross section of the embedded (A) patient specific voice prosthesis and (B) regular cylindrical shaped prosthesis into the reconstructed fistula; and (C) a lateral cross section of a prosthesis-fistula model in accordance with an embodiment of the present disclosure.

FIG. 30 presents a finite element analysis to determine stress concentrations when inward pressure is exerted on the model, simulating a real life scenario of surrounding tissue compression.

FIG. 31 shows minimum and maximum stress experienced by the surrounding tissues under different radial compressions.

FIG. 32 shows a graph of pressure change against airflow rates for varying minimum inner diameters of the voice prosthesis. The flow resistance can be determined by the gradient of the graph. The dotted line denotes the flow resistance of Blom-Singer™ prosthesis, which was used as the benchmark.

FIG. 33 shows a photo of the (A) inner rigid PVC core with the magnetic ball bearing valve and (B) the assembled voice prosthesis with the outer carbon nanocomposite skin in accordance with an embodiment of the present disclosure.

FIG. 34 presents an in-vitro forward flow experimental setup. A is the air pump outlet, R is the variable flow valve, F is the electronic flow meter, P is the pressure transducer, C is the pressure chamber and V is the voice prosthesis.

FIG. 35 presents the schematic diagram of the test rig used to measure the backflow resistance of the voice prosthesis.

FIG. 36 presents the graph of the pressure change against airflow for the different prosthesis from the in-vitro experiment.

FIG. 37 presents a graph of average leakage versus pressure for the patient specific carbon nanocomposite voice prosthesis. The dotted line denotes the maximum quantity of liquid that can be eliminated by the patient through coughing.

FIG. 38 shows photos of an in-vivo porcine animal experiment. (A) The creation of a trachea defect to expose the inner lumen of the trachea; (B) Puncture and insertion of the prosthesis using the MAID; and (C) Voice prosthesis in position.

FIG. 39 presents an evaluation algorithm adopted to optimize the design, fitting and comfort of the voice prosthesis in patients.

### DETAILED DESCRIPTION

In the present disclosure, depiction of a given element or consideration or use of a particular element number in a particular FIG. or a reference thereto in corresponding descriptive material can encompass the same, an equivalent, or an analogous element or element number identified in another FIG. or descriptive material associated therewith. The use of “/” in a FIG. or associated text is understood to mean “and/or” unless otherwise indicated. The recitation of a particular numerical value or value range herein is understood to include or be a recitation of an approximate numerical value or value range, for instance, within +/−20%, +/−15%, +/−10%, or +/−5%.

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). In general, an element of a set can include or be a system, an apparatus, a device, a structure, an object, a process, a physical parameter, or a value depending upon the type of set under consideration.

Herein, reference to automated or semi-automated procedures (e.g., simulation, computational analysis, processing, or algorithm execution) encompasses or can be defined as automated processing by way of a computer system or computing device having a processing unit, a memory, and possibly one or more associated devices such as a set of input devices, a display device, a data storage device. The memory stores program instructions executable by the processing unit, such that processing occurs in accordance with the execution of one or more program instruction sets. The fabrication of one or more types of prosthesis devices or structures in accordance with embodiments of the present disclosure can occur by way of manual and/or automated procedures, such as computer controlled manufacturing (e.g., rapid prototyping/additive manufacturing/3D printing).

In the description that follows, reference to “the invention” shall be construed as reference to an embodiment in accordance with the present disclosure.

### Representative Artificial Trachea

The invention describes a methodology and material for the design, simulation, fabrication and deployment of a carbon nanocomposite tracheal prosthesis, which can be a patient specific carbon nanocomposite tracheal prosthesis. This invention utilizes the simulation of a 3D tracheal model, constructed from volume images or volumetric images of the patient's trachea, together with a prosthesis or prosthesis model, to evaluate and/or aid the design effectiveness and outcome. Such methodology or a similar methodology can also be applied for other tubular tissue replacements such as vascular vessels, nerves and/or intestines.

### Representative Process Flow

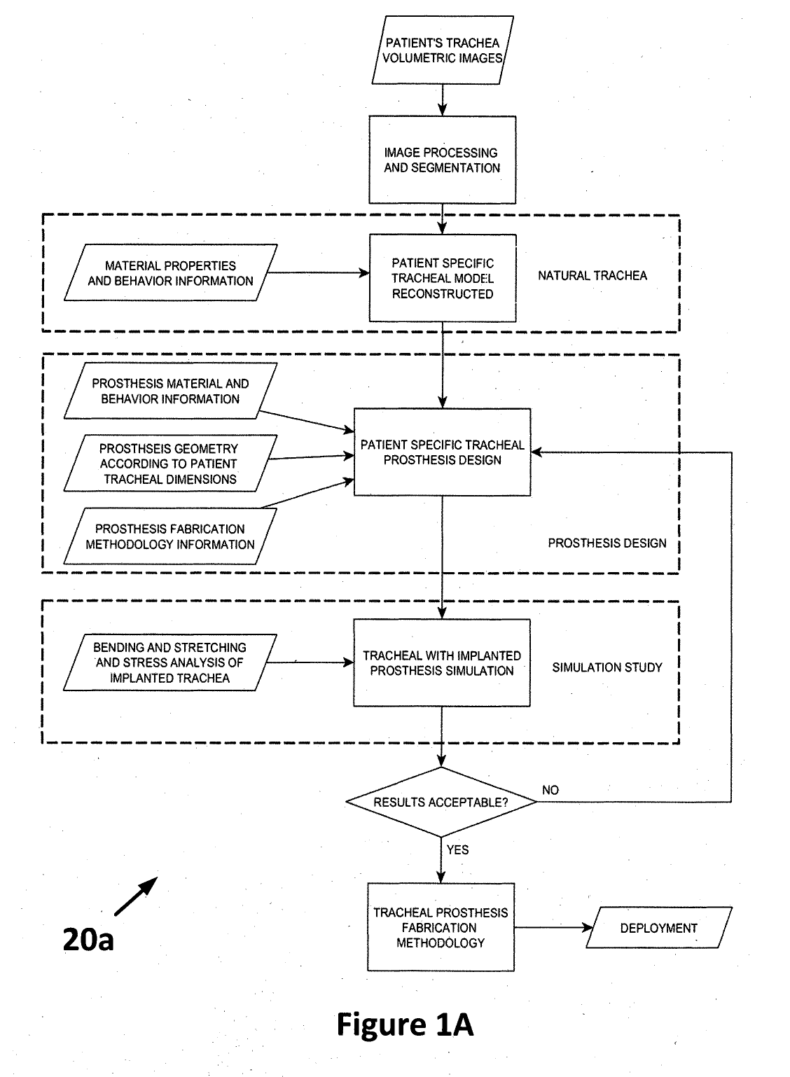

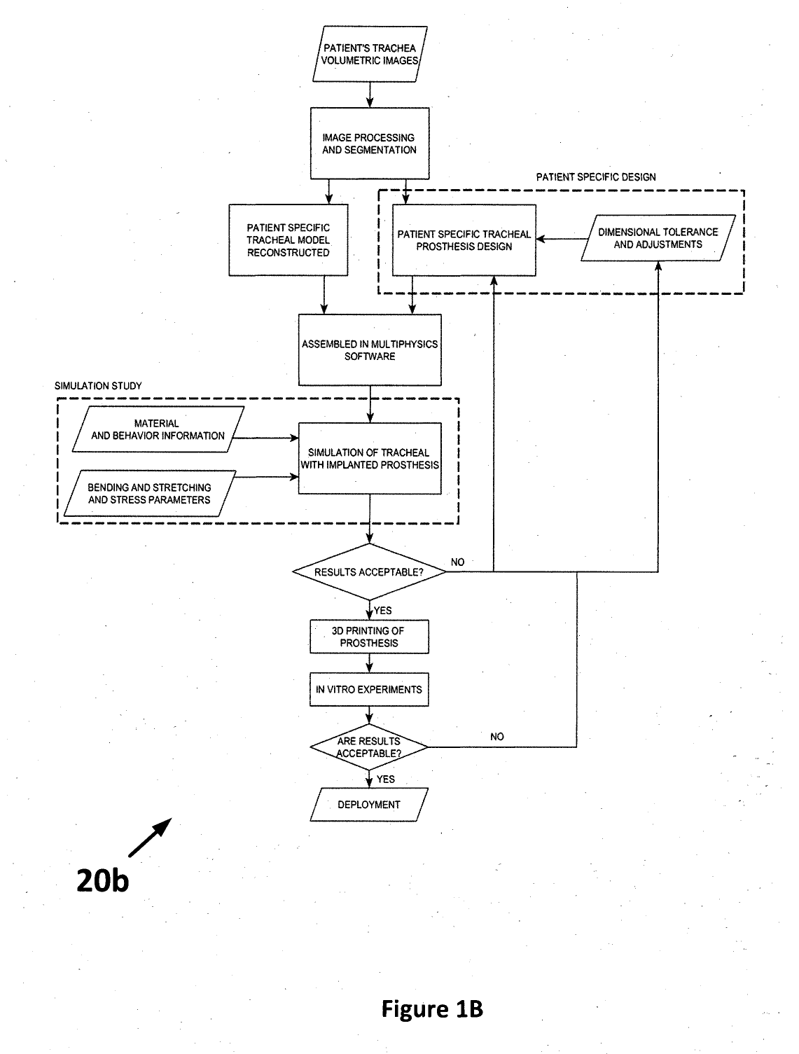

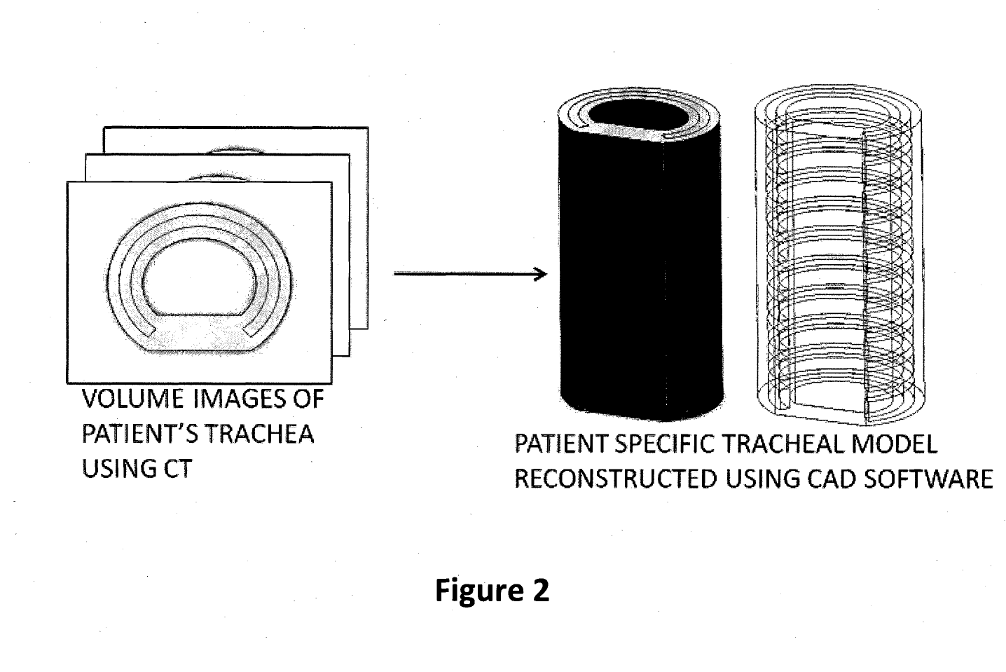

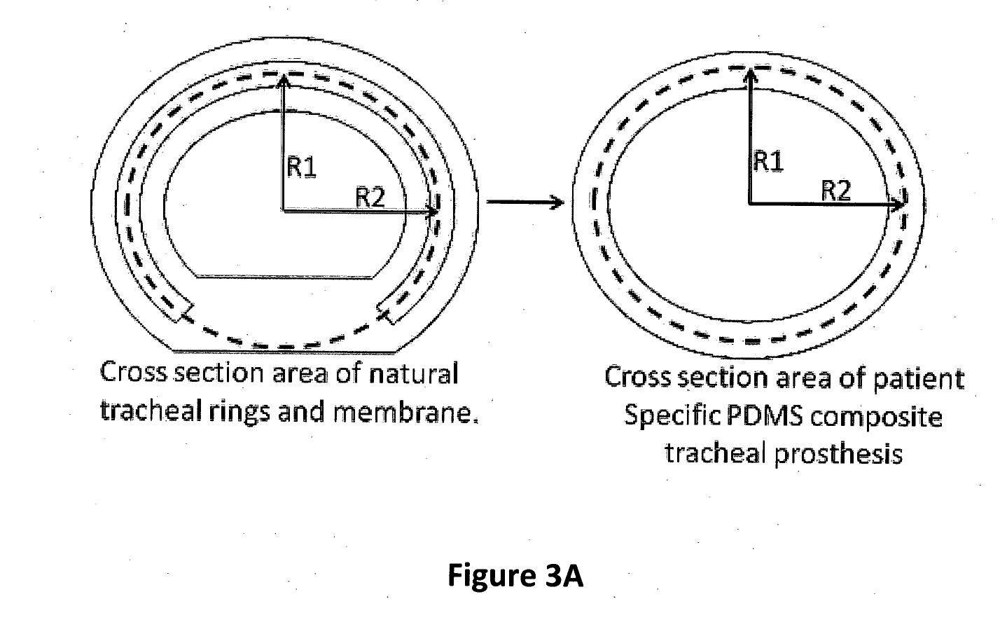

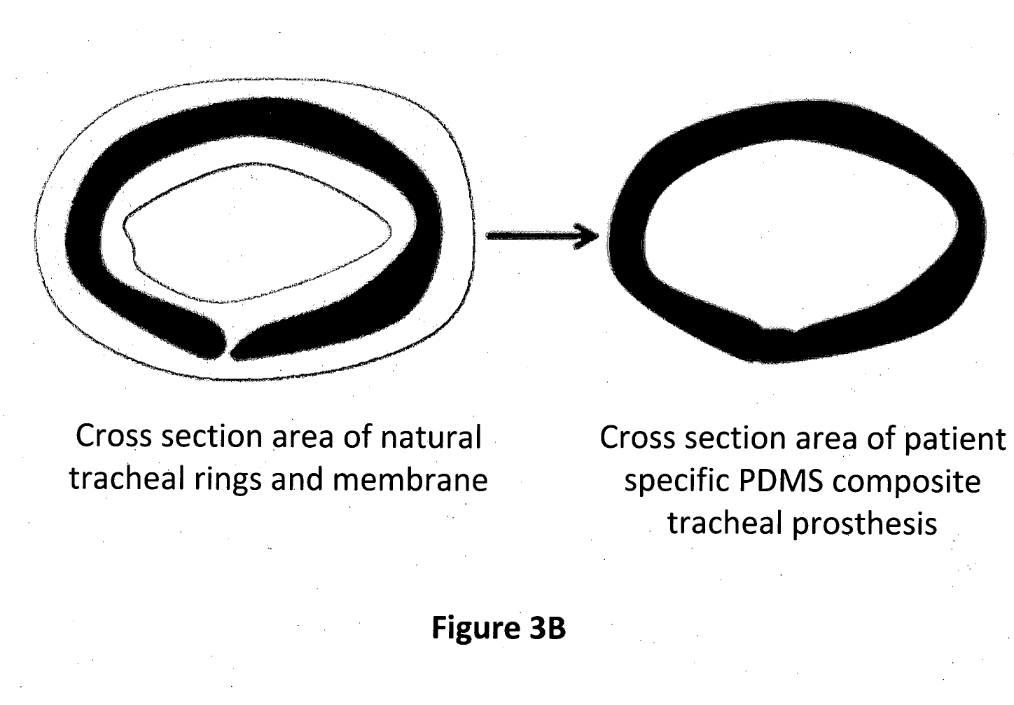

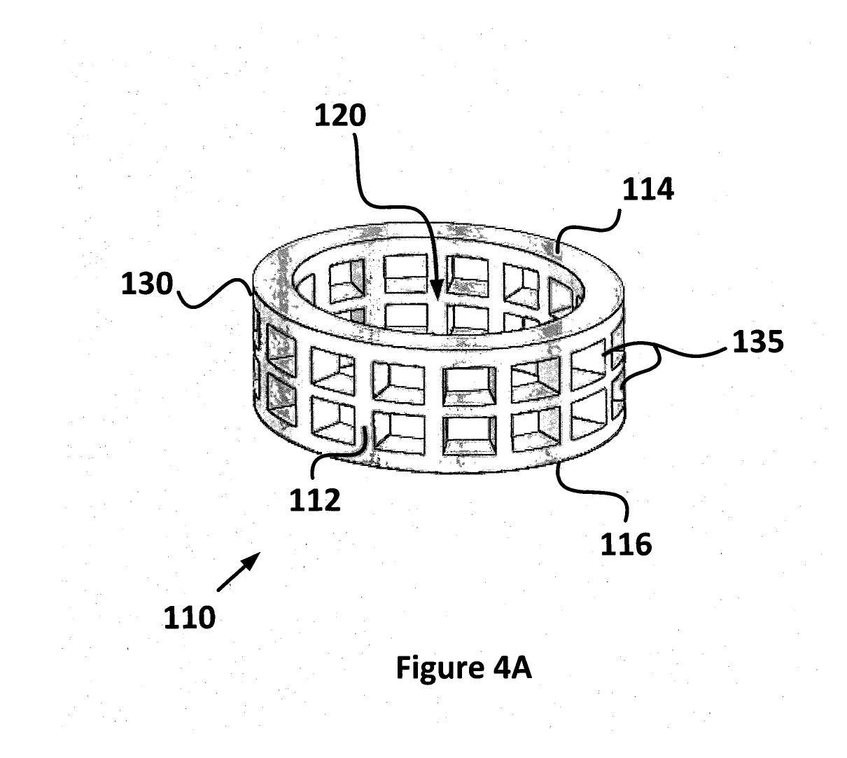

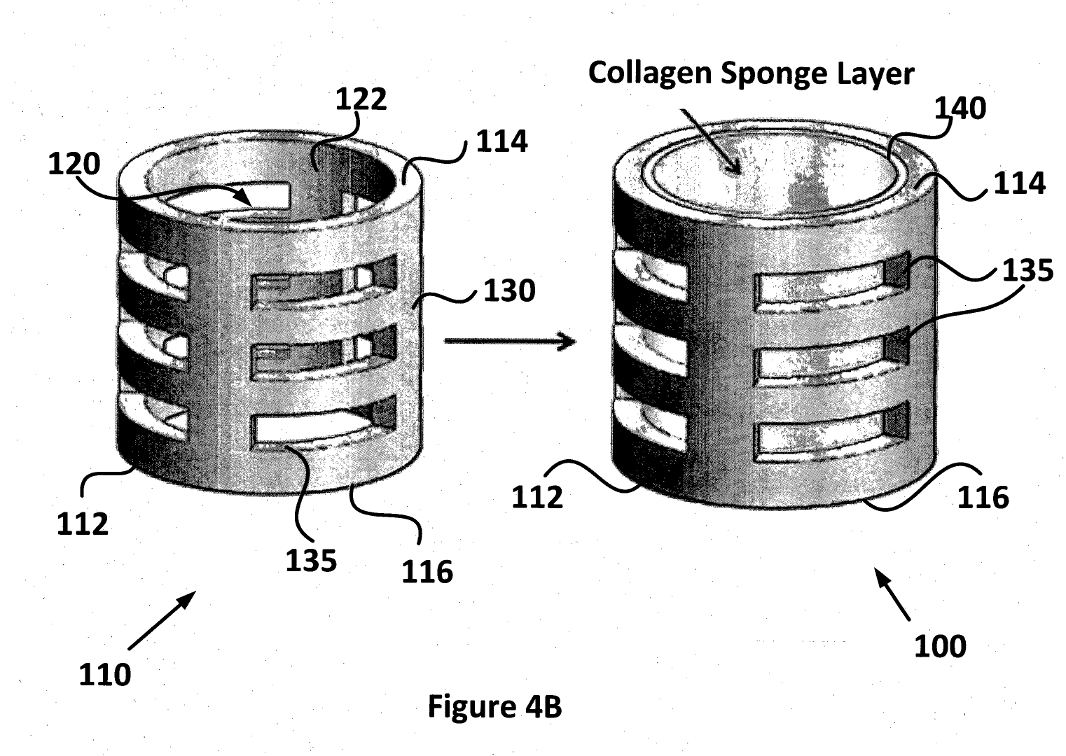

A schematic diagram for a representative flow process is illustrated in FIG. 1A, which shows the stage by stage design and development of the artificial tracheal prosthesis in accordance with an embodiment of the present disclosure. Volume images of the patient's trachea in an axial direction are first obtained via CT scan or MRI. After which, these images will undergo processing and segmentation in order to reconstruct the full 3D tracheal model structure (FIG. 2). The material properties of the cartilage rings and membrane based on current information are input into their respective regions in the model. The next stage is the prosthesis design stage, where the patient specific tracheal prosthesis is designed according to the dimensions of the elliptic tracheal cartilage rings (FIG. 3A). The thickness of the membrane from the CT images may be used as a gauge to determine the thickness of the biodegradable matrix. Materials suitable for the non-biodegradable scaffold and the biodegradable matrix are then selected based on current knowledge of their mechanical properties and biochemical properties. For this invention, carbon nanotube fibers embedded in PDMS matrix were selected for the scaffold, while Type I collagen sponge was selected for the matrix. Materials must also be biocompatible and able to be sterilized. The design of the scaffold also needs to consider the ease of fabrication. In several embodiments, the final design after several simulations is an elliptic and hollow tubular structure with horizontal holes (FIG. 4A).

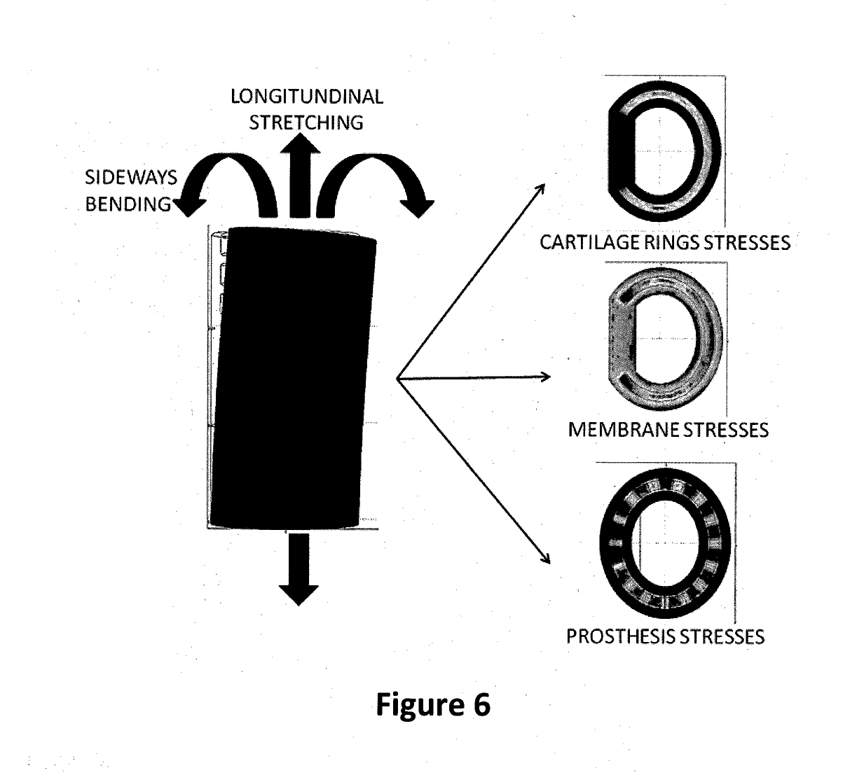

The next stage, is the combination of the constructed tracheal model with the implanted prosthesis scaffold to simulate the final end goal whereby the entire collagen matrix has been degraded and the PDMS composite scaffold is left embedded in the newly ingrown membranous tissue. Bending and stretching motions similar to daily trachea activities are simulated on the model to study the stress concentrations of the surrounding cartilage rings, membrane and prosthesis (FIG. 6). In this invention, 316L stainless steel material and a modular circular shape design prosthesis was also simulated to study the difference (if any) on the stress concentration levels and whether the determined configuration was better.

In the event of acceptable results, the following stage is the fabrication methodology of the artificial trachea. Generally, the use of 3D printing can be employed to fabricate the user centric mold for the curing of PDMS composite. Following which, an aluminum mold can be machined out for the baking and dry freezing of the collagen sponge matrix together with the cured PDMS composite scaffold. After successful fabrication and satisfied quality control checking, the final stage of the schematic diagram is the deployment stage which includes implantation the prosthesis into the defective trachea of an animal or human subject/patient.

### Representative Construction of Patient Specific Tracheal Model

In one aspect, the invention provides a methodology for the evaluation of the performance quality of the designed tracheal prosthesis in a simulated patient specific tracheal environment. This initial stage of the invention focuses on the recreation of the patient specific tracheal model. The model is based on volume images captured from the patient's trachea via one or more types of clinical devices such as x-ray, magnetic resonance imaging (MRI), ultrasound techniques (US), computerized tomography (CT) scanners, rotational angiography and/or other imaging modalities in the axial direction. Preferably, the patient being scanned should be the one suffering from a tracheal disorder or disease like stenosis or cancer, and requires treatment. This is to replicate the characteristics of dysfunctional/diseased tracheal tissue for more realistic simulation of the deployment and performance evaluation of the designed prosthesis. These 2D images can be constructed into a 3D model as shown in FIG. 2. These captured images are then processed using image processing tools like Photoshop™ or MATLAB™ whereby the contour and outline of each image is extracted and stacked on top of each other to forma 3D tracheal model. The 3D model is reconstructed in computer aided design (CAD) software like Solidworks™. Segmentation can also be deployed as part of image processing to reconstruct the specific geometry of the trachea. A tracheal model constructed for the purpose of a representative embodiment in accordance with the present disclosure is 58 mm long and has 10 cartilage rings, each of 4 mm thickness and spacing of 2 mm between rings. As the trachea consists primarily of 2 different tissues, namely the membranous tissues and the cartilage rings, each with their own different biomechanical properties and behaviors; such parameters relating to their properties based on known information can be input into their respective regions for simulation purposes. Although recent research has utilized the elastic Neo Hookean model for tracheal cartilage behaviour and Holzapfel strain energy of two orthogonal families of collagen fibres for the membranous tissue, these models were invalid for a 3D solid tracheal model undergoing stretching and bending motions due to their inherent assumptions of incompressibility. The model utilized in this invention takes the mechanical properties of the cartilage ring to be homogenous (Trabelsi 2010) and linearly elastic for strains of up to 10% with minimal residual strains (Rains 1992). For the model of the mucosa membrane, literature research has shown that the membrane behaves differently in the longitudinal and transverse directions, hence the membrane was modelled as an orthogonal behavioral material with different Young Modulus (E), Shear Modulus (G) and poisson ratio (v) in all three principle axis according to values from research (Sarma 2003). The values used are as follows:

Trachea cartilaginous rings: E=3.33 MPa; v=0.49
Mucosa membrane: Eₓₓ (longitudinal direction)=0.36 MPa; G_{xy}=0.124 MPa; v_{xy}=0.45
E_{yy} (transverse direction)=0.3 MPa; G_{xz}=0.124 MPa; v_{xz}=0.375
E_{zz} (radial direction)=0.3 MPa; G_{yz}=0.124 MPa; v_{yz}=0.375

### Representative Patient Specific Tracheal Prosthesis Design

This section describes a representative design methodology for the patient specific tracheal prosthesis. The non-biodegradable scaffold of the prosthesis will serve as a mechanical backbone support structure, equivalent to the cartilage rings, when implanted. Its design should also consider the ease of manufacturability (e.g., via a molding process) while having holes through its surface to allow for membranous tissue ingrowth. With these considerations, the final design of the tubular elliptical prosthesis, after considerations of multiple suitable candidates, is illustrated in FIG. 4. Based on the collated 2D images of the patient, the geometry of the diseased portion to be exercised can be used to determine the dimensions of the prosthesis (FIG. 3). As the natural cartilage rings are almost elliptic in shape, the radius of the major and minor axes can be approximately measured from the average of the inner and outer circumference of the D-shaped ring. These values are then used for the dimensions of the non-biodegradable scaffold of the prosthesis as it will be effectively replacing the native cartilage rings when implanted.

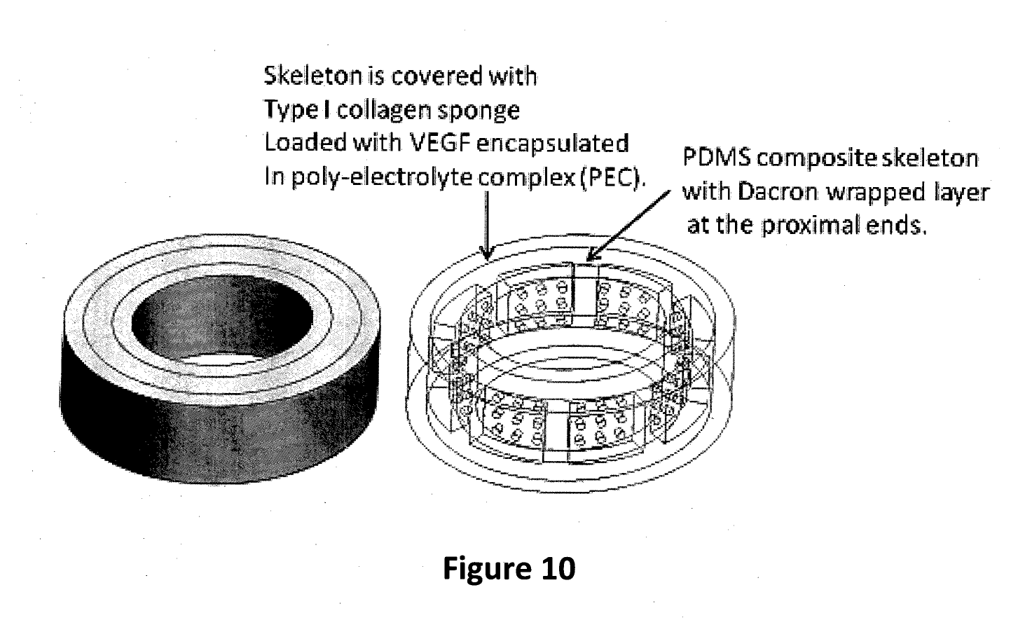

The thickness of the membranous tissue surrounding the cartilage rings can also be used as a gauge to determine the thickness of the collagen sponge matrix required to cover the non-biodegradable scaffold. The final artificial trachea prosthesis is a multi-material device (FIG. 10). Its inner non-biodegradable scaffold is typically or most suitably made of a PDMS-carbon nanotube composite, due to (a) the ability to tailor the mechanical properties to make it similar or very similar to the patient's native tracheal rings, and (b) its biocompatibility. Hence it is a suitable material to prevent buckling of the lumen and stenosis due to external pressure, while allowing for some degree of bending and stretching. The proximal ends of the tubular prosthesis are wrapped with a layer of Dacron to make it leak proof, while providing a strong surface to suture on. Next, the PDMS composite skeleton is coated with a thick layer of freeze-dried solidified Type I collagen sponge on both the inner and outer sides. This biodegradable layer serves as a temporary matrix for the in growth of cells and blood vessels into the prosthesis, while keeping the device air tight when it is soaked in the patient's blood medium. Furthermore, since collagen Type I is essentially a majority component of the surrounding membranous tissue, it will be easily integrated and promotes more efficient tissue in-growth. The collagen matrix is also loaded with the protein Vascular Endothelial Growth Factor (VEGF) just before implantation, which helps to stimulate and accelerate the ingrowth of blood vessels and cells into the prosthesis. The VEGF can also be encapsulated in Poly Electrolyte Complex (PEC) to prolong its lifespan in vivo. During the following few days and weeks after surgery, the collagen matrix will degrade gradually to make space for more tissue in growth, until the entire PDMS scaffold is covered by the newly ingrown natural tracheal membrane.

### Representative Simulation Study Methodology

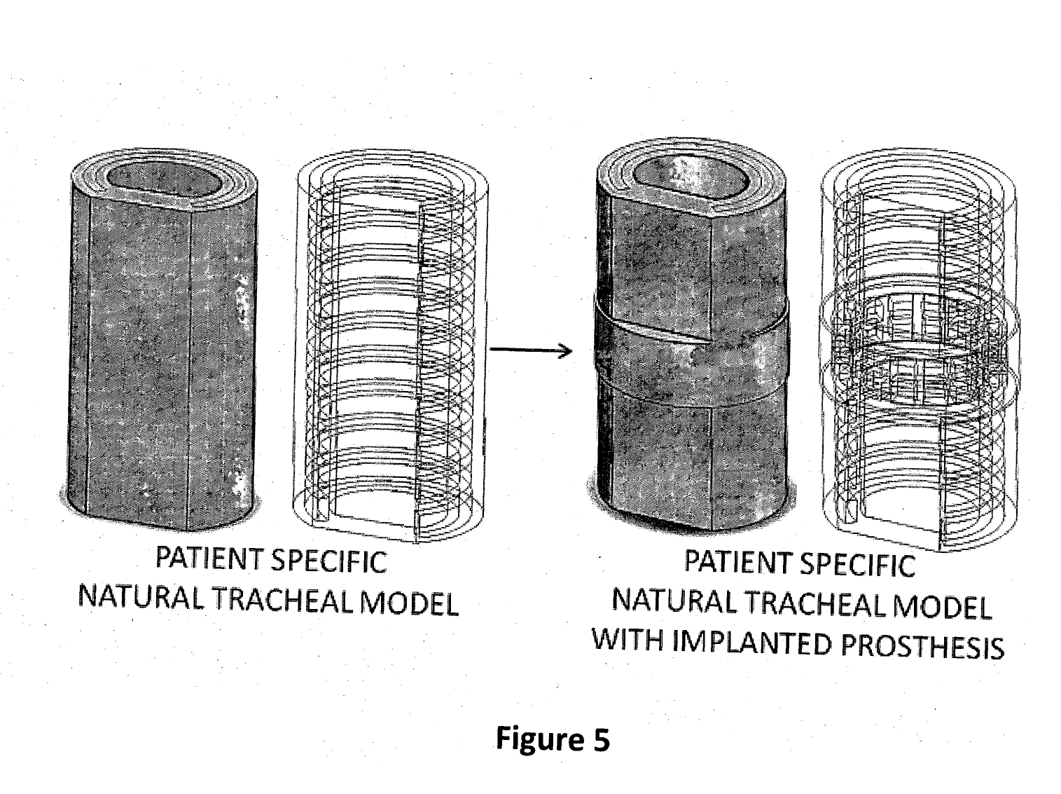

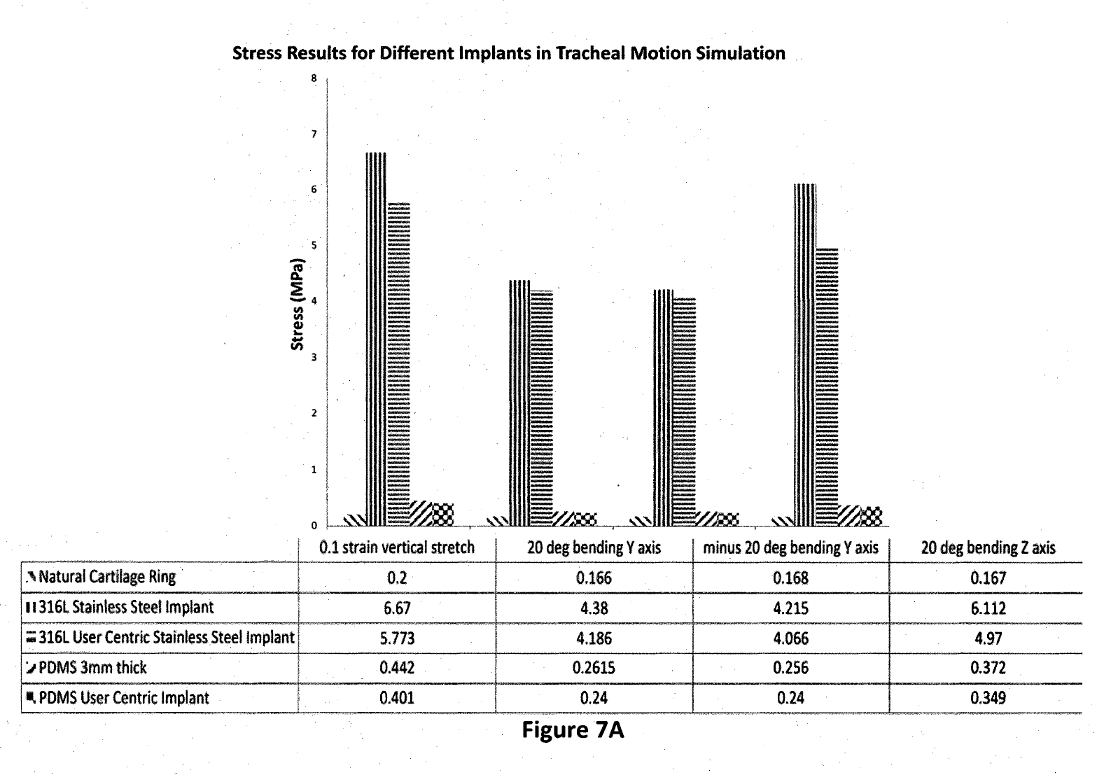

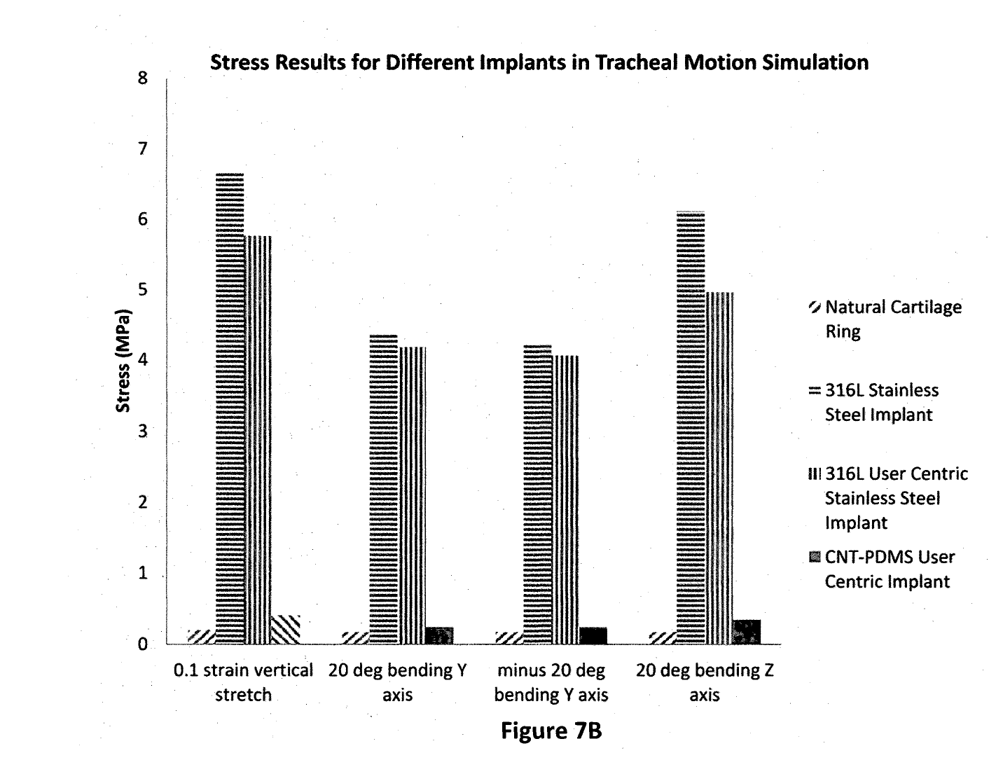

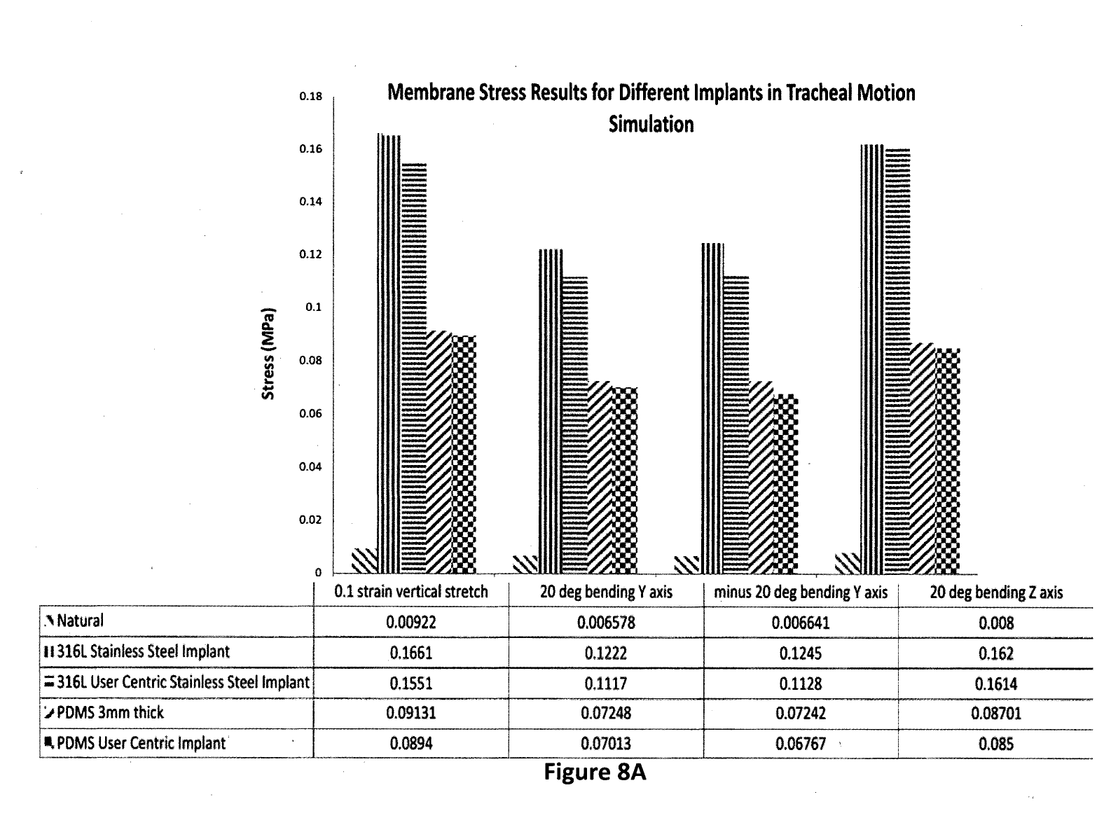

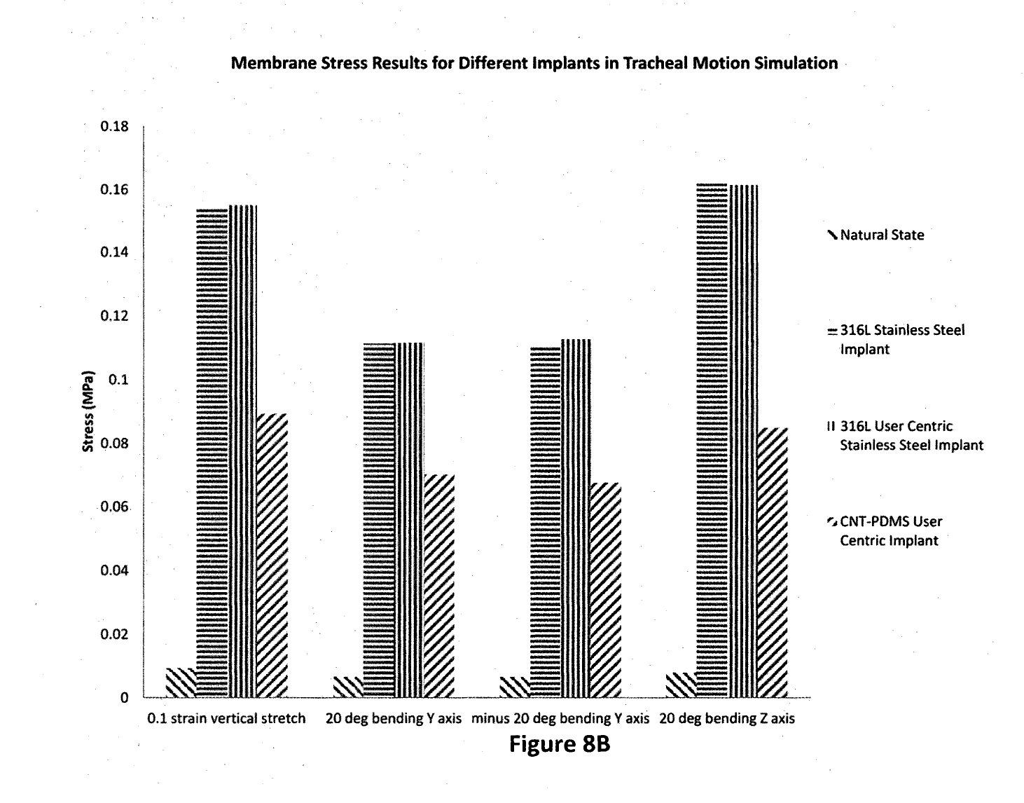

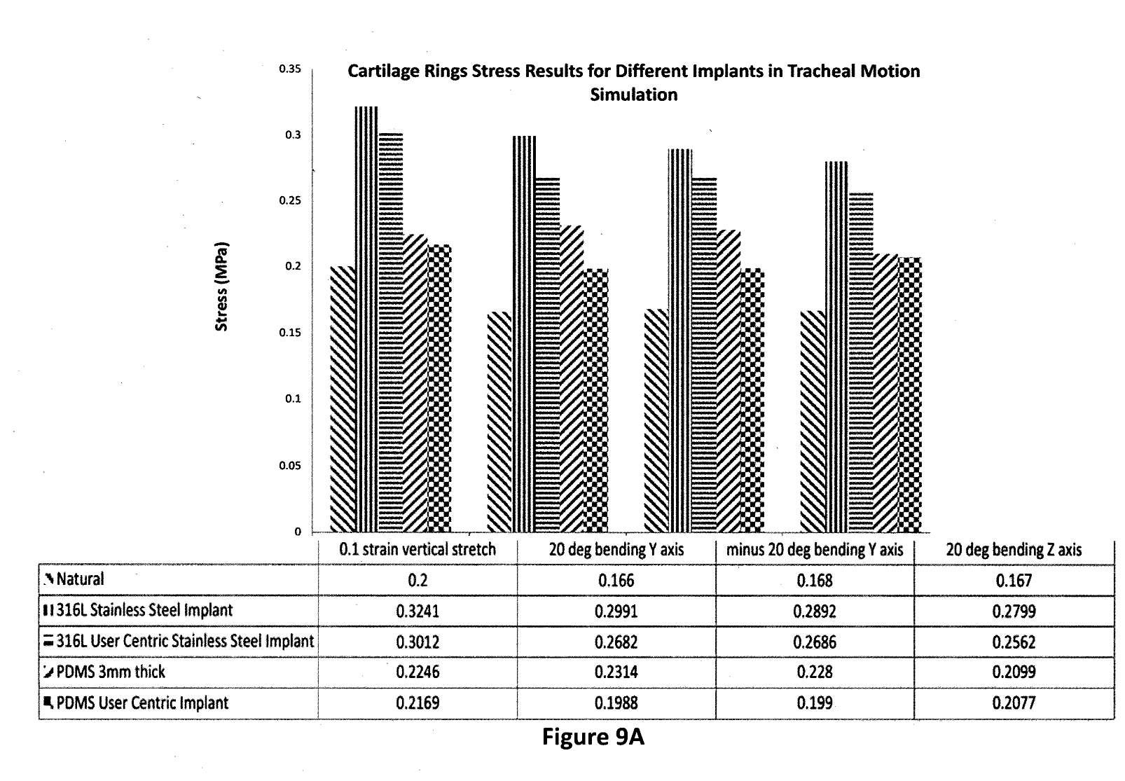

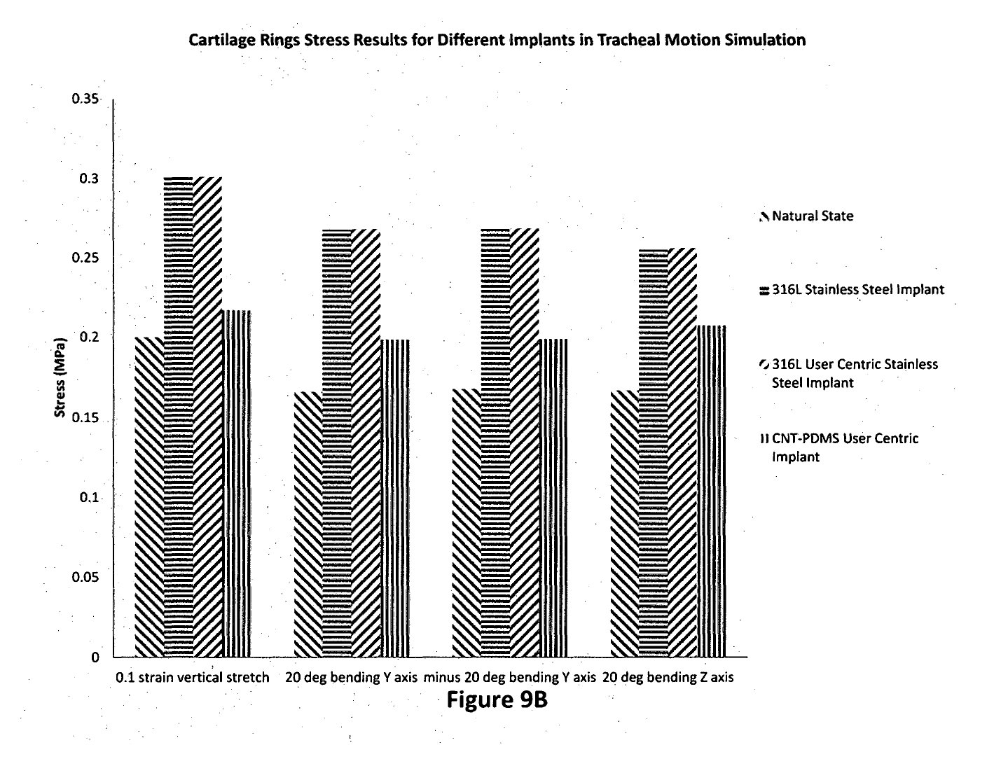

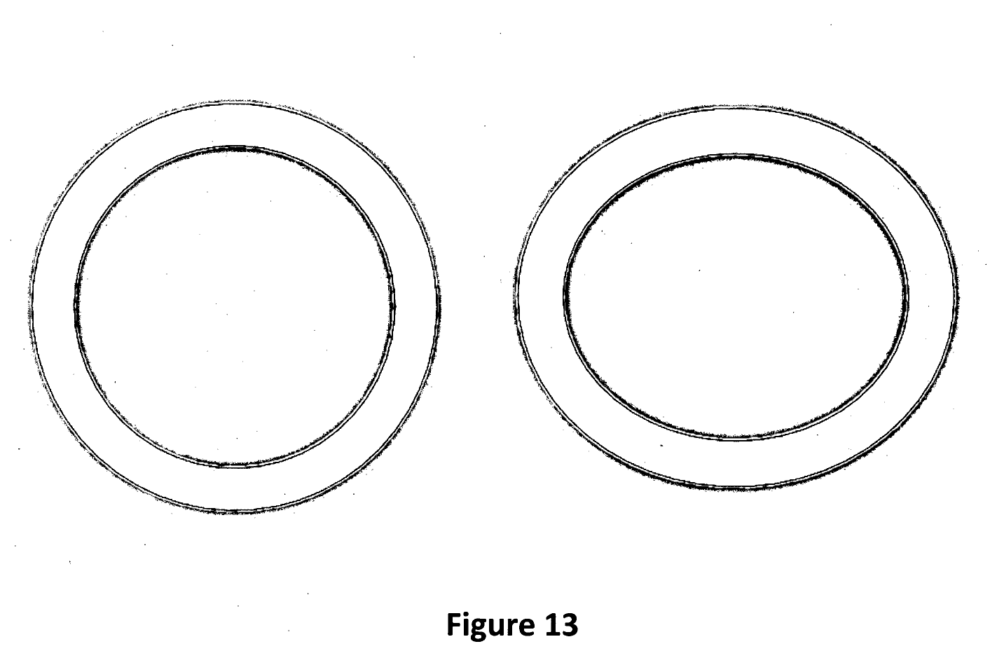

This section describes the integration of the tracheal prosthesis into the constructed patient specific tracheal model for simulation and performance evaluation. In addition to PDMS composite, properties of 316L stainless steel was also used as a scaffold material for simulation and comparison, due to its strength and ability to withstand compressive forces in the tracheal loading conditions, as well as its biocompatibility in the host body. Using Solidworks™ assembly module, cartilage rings from the diseased portion will be removed and replaced with the designed scaffold. In this simulation study, the 5^{th} and 6^{th} cartilage rings from the top of the tracheal model were removed and replaced with the designed scaffold (FIG. 5). This is to simulate a real life scenario, whereby the entire collagen matrix of the implant has been broken down and only the scaffold is left embedded in the newly formed membranous tissues, which takes over the function of the removed cartilaginous rings. For comparison purposes in the simulation, both modular circular shaped hollow prosthesis and elliptical shaped hollow prostheses (FIG. 13) were designed using Solidworks™. Material properties of both 316L stainless steel and PDMS composite are also input into both geometrical designs for results comparisons. The assembly was put through stretching and bending motions in COMSOL™ Multi-physics to study the stress concentrations in the different regions during daily motions of the trachea. For this invention, stretching by 10% strain (due to swallowing) and bending in the Y and Z directions (from head and heck movements) were done on both natural and augmented trachea to study the effect of the implant on the surrounding natural membrane and cartilage rings. Results were taken from the maximum stress in the surrounding membrane and from the closest cartilage ring and tabulated in a table for comparison. From the results (FIG. 7, FIG. 8, FIG. 9), PDMS composite was confirmed to be a suitable material or the material of choice for the scaffold due to closer similarities in stress concentrations to the native tracheal rings and membrane. Furthermore, results data also pointed out a reduction in stress concentrations when a user centric design is used rather than a modular geometrical shape.

### Representative Manufacturing Methodology

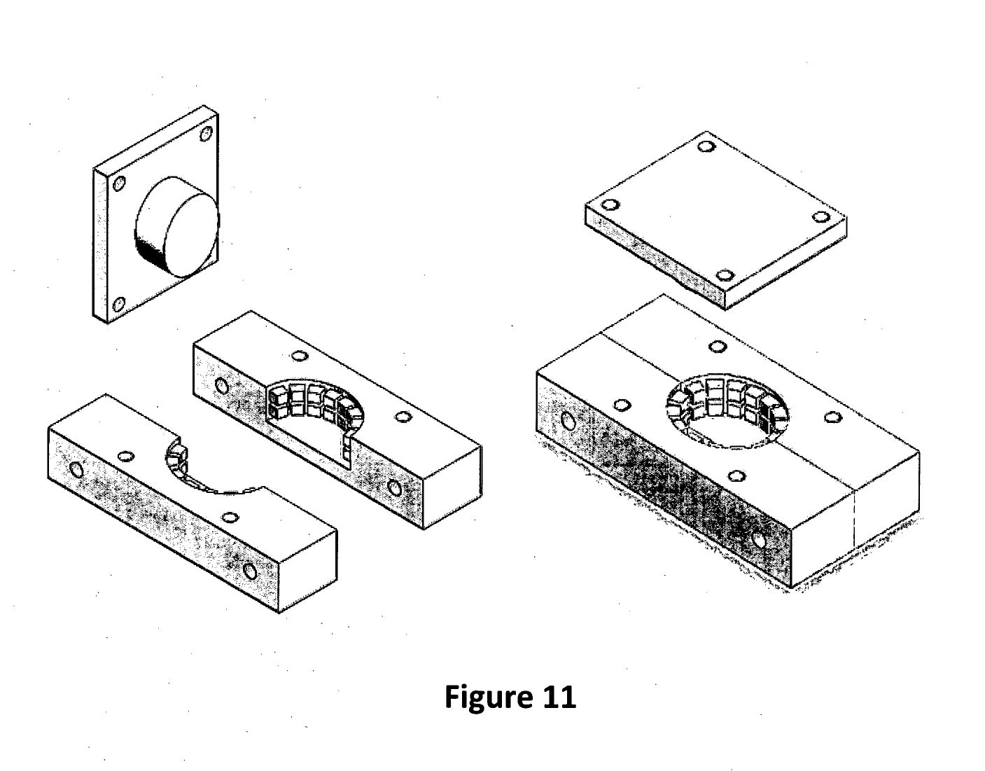

This section relates to a methodology for the fabrication of a patient specific artificial tracheal prosthesis. The design of the non-biodegradable PDMS scaffold has taken into account the mode of its fabrication, which in an embodiment would be by way of mold forming. A mold for the PDMS is firstly rapid prototyped out according to the dimensions and shape of the patient's tracheal rings (FIG. 11). The design of the mold is such that it includes two base parts joint together with a removable top cover with a central mold. This is to facilitate easier removal of the pure PDMS as the mold can be dismantled part and assembled. Care must be taken in the design of the mold for shrinkage allowance of the cured PDMS. Sylgard 184™ brand is used as the PDMS material and it is mixed thoroughly in a ratio of 5:1 with its curing agent to achieve optimal strength according to product information. Carbon nanotubes are then added into the slurry and stirred thoroughly. The mixture is then poured into the base mold and left in a vacuum jar or desiccator for 20 minutes to remove any trapped bubbles which would affect the mechanical properties of the final product. Following which, the top cover with the central mold is placed carefully into the base mold to avoid the formation of any new bubbles. Excess PDMS solution that is displaced during the insertion of the top mold should be cleared away. The entire mold, with its constituents, is left to cure at room temperature (25° C.) for 48 hours. After curing, the mold is dismantled apart and the cured component is wrapped at its proximal ends with Dacron material.

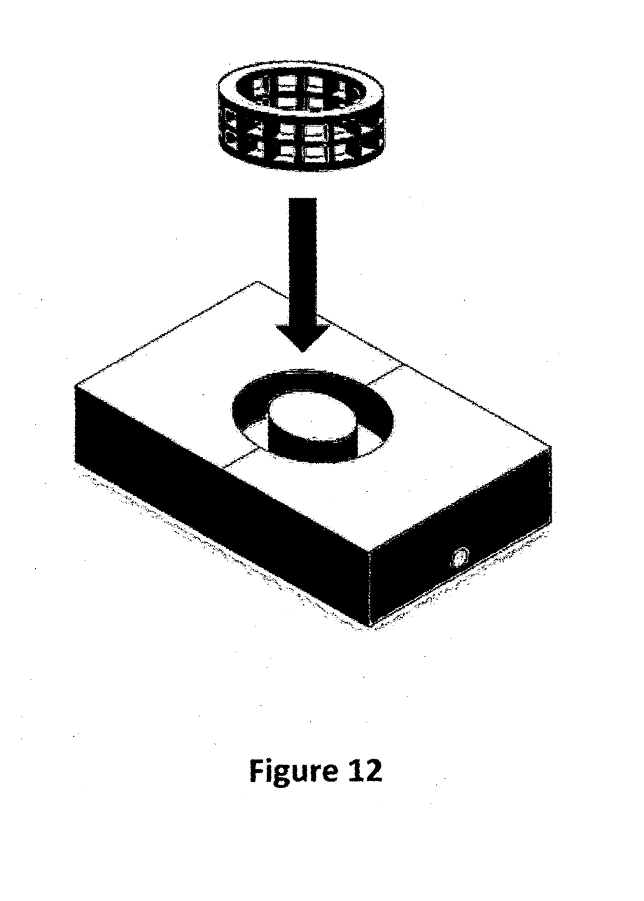

The fabrication of a second mold is undertaken, for the formation of the Type I collagen sponge matrix around the scaffold. The design of the mold can be seen in FIG. 12. The width of the hollow section of the mold would determine the thickness of the collagen matrix to be produced. The cured PDMS bio-composite scaffold is first placed into the hollow region, which is shaped as an ellipse as well. Type I collagen (e.g., porcine) solution is dissolved in aqueous hydrochloric acid (pH 3) to give a final concentration of 1% by weight (Yamashita 2007). This is followed by homogenizing of the solution at 8000 rpm for 15 minutes. This solution is then poured carefully into the mold cavity containing the scaffold and the entire mold is placed into a freeze drier at −80° C. After which, the mold is placed into a vacuum oven at 140° C. for 12 hours for cross linking to occur. Upon completion, the artificial tracheal should be removed from the mold and sealed in an air tight plastic packaging for storage purposes.

### Representative Deployment Methodology

This section describes details for the deployment of the artificial tracheal prosthesis in a living organism. During the operation, the surgeon should first remove the artificial trachea prosthesis from its plastic storage packaging in a sterile condition. The surgeon should then proceed with the resection of the diseased portion of the patient's tracheal. Ideally, the length of the prosthesis should be around or approximately the same length of the resection portion, which could be determined during the initial diagnostic imaging stage. After resection, a mechanical ventilator tube should be placed into the lower exposed trachea end to provide oxygen supply to the lungs. The next step would be to dip the prosthesis into a prepared solution of PEC-encapsulated VEGF or VEGF, and ensure that the solution is absorbed as evenly as possible through the entire device. After which, the artificial trachea is immersed into the patient's own blood medium and the collagen sponge will be rendered air tight after it soaks up the blood. The tubular prosthesis is then joined and sutured to the exposed native trachea to bridge the gap. Care should be taken to ensure that the suturing should be done on the Dacron layer or bio-composite scaffold and not on the collagen matrix as the latter interface might not have sufficient mechanical strength to withstand the anastomotic tension. Finally, the patient is sewed up.

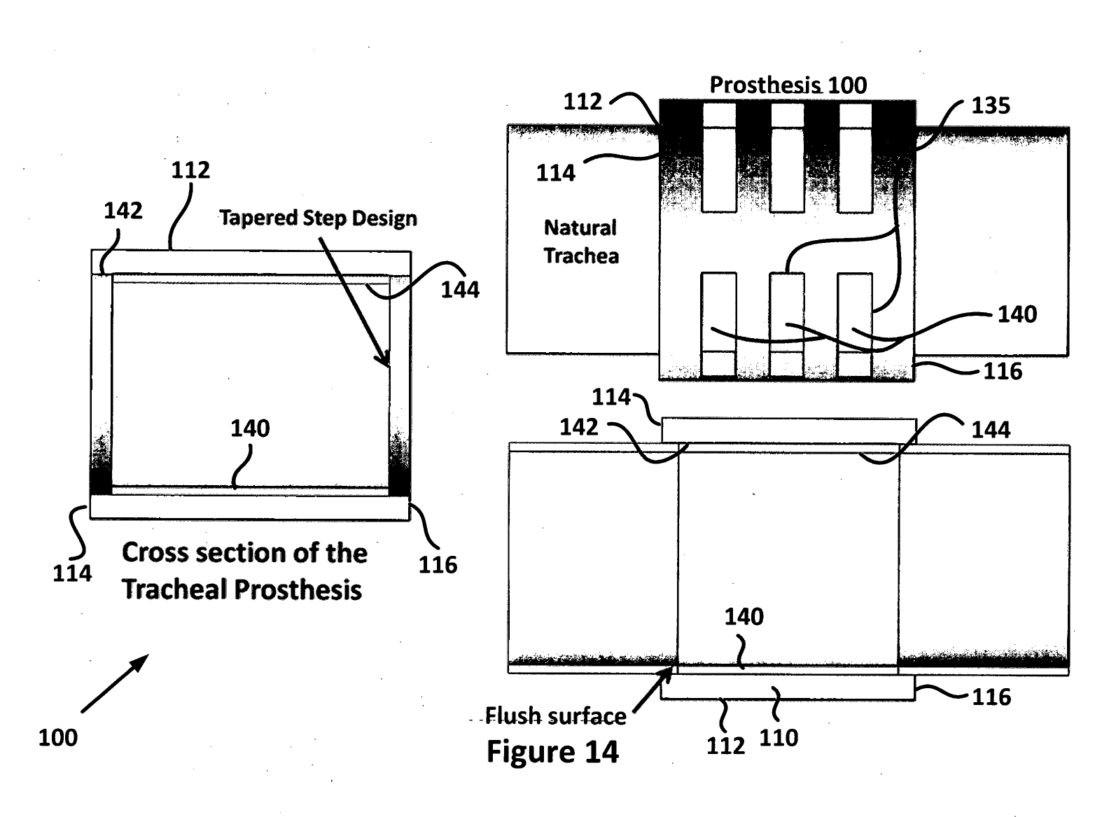

The schematic diagram for the invention flow process, in FIG. 1B, shows an iterative algorithm used in the development of the artificial tracheal prosthesis in accordance with a second embodiment of the present disclosure. Volume images of the patient's trachea in an axial direction are first obtained via CT scan or MRI. After which, these images undergo processing and segmentation in order to reconstruct the full 3D patient specific tracheal model structure (FIG. 2). The model is useful in the simulation studies of the prosthesis design. In addition, the patient specific tracheal prosthesis is designed according to the dimensions of the elliptic tracheal cartilage rings (FIG. 3B). The natural cartilage rings can be easily distinguished from the surrounding membrane from the images and their irregular elliptic shape are processed and are used to form the skeleton of the prosthesis. Materials suitable for the non-biodegradable scaffold skeleton and the biodegradable matrix are then selected based on current knowledge of their mechanical properties and biochemical properties. Carbon nanotubes fibers embedded in PDMS matrix was selected for the skeleton, while Type I collagen sponge was selected for the lumen coating. Materials must also be biocompatible and sterilizable. The design and material of the scaffold also needs to also consider the mode of fabrication and intended function in-vivo. Firstly, since the mode of fabrication in this case is via 3D-printing due to the intrinsically complex shape and geometry of the trachea prosthesis, materials compatible with rapid prototyping like thermoplastics should be selected. Secondly, the skeleton's purpose is to maintain airway patency under compression stresses while still being flexible enough for motion. Thirdly, since cellular in-growth is required for the success of the implant, the scaffold should have sufficient holes to allow in-growth while still maintaining its structural strength. The final design after several iterations was an elliptic and hollow tubular structure with horizontal holes (FIG. 4B). A tapered design was implemented near the proximal ends of the prosthesis (FIG. 14). The tapered step due to the layer of collagen sponge in the skeleton lumen allows for the two joining surfaces to be flushed with one another during anastomosis. This helps guide the migration of the ciliated epithelium into the lumen of the prosthesis to aid in mucous removal.

The next stage is the combination of the constructed tracheal model with the implanted prosthesis scaffold to simulate the final end goal whereby the entire collagen matrix has been degraded and the nanocomposite scaffold is left embedded in the newly ingrown membranous tissue. Bending and stretching motions similar to daily trachea activities are simulated on the model to study the stress concentrations of the surrounding cartilage rings, membrane and prosthesis (FIG. 6). 316L stainless steel material and a modular circular shape design prosthesis was also simulated to study the difference (if any) on the stress concentration levels and whether the determined configuration was better.

In the event of acceptable results, the following stage is the fabrication methodology of the artificial trachea. Generally, the use of 3D printing is employed to fabricate the patient specific nancomposite prosthesis. Following which, mold such as an aluminum mold can be machined out for the dry freezing and baking of the collagen sponge layer that will be attached to the lumen of the prosthesis. After successful fabrication and quality control checks, the scaffold can be subjected to in-vitro testing and finally deployed. In some situations, deployment can involve implantation in a porcine model to evaluate its effectiveness.

### Representative Reconstruction of Patient Specific Tracheal Model

In one aspect, the invention provides a methodology for the evaluation of the performance quality of the designed tracheal prosthesis in a simulated patient specific tracheal environment. This initial stage of the invention focuses on the recreation of the patient specific tracheal model. The model is based on volume images captured from the patient's trachea via clinical devices such as x-ray, magnetic resonance imaging (MRI), ultrasound techniques (US), computerized tomography (CT) scanners, rotational angiography or other imaging modalities in the axial direction. Preferably, the patient being scanned should be the one suffering from tracheal disorder like stenosis or cancer, and requires treatment. This is to replicate the characteristics of a diseased tracheal tissue for more realistic simulation of the deployment and performance evaluation of the designed prosthesis. These 2D images can be constructed into a 3D model as shown in FIG. 2. These captured images are then processed using image processing tools like Photoshop™ or MATLAB™ whereby the contour and outline of each image is extracted and stacked on top of each other to form a 3D tracheal model. The 3D model is reconstructed in computer aided design (CAD) software like Solidworks™. Segmentation can also be deployed as part of image processing to reconstruct the specific geometry of the trachea. The tracheal model constructed for the purpose of this invention is 58 mm long and has 10 cartilage rings, each of 4 mm thickness and spacing of 2 mm between rings. As the trachea consists primarily of 2 different tissues, namely the membranous tissues and the cartilage rings, each with their own different biomechanical properties and behaviors; such parameters relating to their properties based on known information can be input into their respective regions for simulation purposes. Although recent research has utilized the elastic Neo Hookean model for tracheal cartilage behaviour and Holzapfel strain energy of two orthogonal families of collagen fibres for the membranous tissue, these models were invalid for a 3D solid tracheal model undergoing stretching and bending motions due to their inherent assumptions of incompressibility. The adopted model in this invention takes the mechanical properties of the cartilage ring to be homogenous (Trabelsi 2010) and linearly elastic for strains of up to 10% with minimal residual strains (Rains 1992). For the model of the mucosa membrane, literature research has shown that the membrane behaves differently in the longitudinal and transverse directions, hence the membrane was modelled as an orthogonal behavioral material with different Young Modulus (E), Shear Modulus (G) and poisson ratio (v) in all three principle axis according to values from research (Sarma 2003). The values used are as follows:

Trachea cartilaginous rings: E=3.33 MPa; v=0.49
Mucosa membrane: Eₓₓ (longitudinal direction)=0.36 MPa; G_{xy}=0.124 MPa; v_{xy}=0.45
E_{yy} (transverse direction)=0.3 MPa; G_{xz}=0.124 MPa; v_{xz}=0.375
E_{zz} (radial direction)=0.3 MPa; G_{yz}=0.124 MPa; v_{yz}=0.375

### Representative Patient Specific Tracheal Prosthesis Design

This section describes the design methodology for the patient specific tracheal prosthesis. The non-biodegradable skeleton of the prosthesis will serve as a mechanical backbone support structure, equivalent to the cartilage rings, when implanted. Its design should also consider the ease of manufacturability (via 3D printing). It should have sufficient holes for vascularization and tissue in growth while still maintaining its structural strength. With these considerations, the final design of the tubular patient specific prosthesis, after considerations of multiple suitable candidates, can be found in FIG. 4B. Based on the collated 2D images of the patient, the geometry of the diseased portion to be exercised can be used to determine the dimensions of the prosthesis (FIG. 3B). As the natural cartilage rings are almost elliptic in shape and are sometimes irregular, the cartilage rings should be differentiated from the membranous tissue via-image processing and extracted for the skeletal design of the prosthesis. The thickness of the natural trachea may be used as a gauge to determine the thickness of the collagen sponge layer that coats the lumen of the skeletal scaffold.

The final artificial trachea prosthesis is a multi-material device (FIG. 4B). Its non-biodegradable skeleton is preferably made of CNT-PDMS nanocomposite, due to the latter's ability to tailor its mechanical properties to that of the native tracheal rings and its biocompatibility. Hence it is a suitable material to prevent buckling of the lumen and stenosis due to external pressure, while allowing for some degree of bending and stretching. The large horizontal holes in the skeleton allows for quick vascularization and tissue in growth to assimilate the prosthesis into the body. A biodegradable layer of type I collagen sponge in the inner lumen of the skeleton serves as a temporary matrix to guide tracheal epithelial cells migration into the prosthesis, while keeping the device air tight when it is soaked in the patient's blood medium. Furthermore, since collagen Type I is essentially a majority component of the surrounding membranous tissue, it will be easily integrated and promotes more efficient tissue in-growth. A step tapered step design is incorporated into the prosthesis (FIG. 14). This allows for the two adjacent lumen surfaces to be flushed with one another when the resected end of the natural trachea is inserted into the lumen of the prosthesis. Through this design, the collagen layer can guide the migration of the ciliated epithelium from the lumen surface of the trachea into the prosthesis to perform its function of mucous removal. The collagen matrix is also loaded with the protein Vascular Endothelial Growth Factor (VEGF) and human Endothelial Growth Factor (hGF) just before implantation, which helps to stimulate and accelerate angiogenesis and vascularization of the prosthesis. During the following few days and weeks after surgery, the collagen matrix will degrade gradually to make space for more tissue in growth, until the entire CNT-PDMS skeleton scaffold is covered by the newly formed tracheal membrane.

### Representative Simulation Study Methodology

This section describes the integration of the tracheal prosthesis into the constructed patient specific tracheal model for simulation and performance evaluation. In addition to PDMS composite, properties of 316L stainless steel was also used as a scaffold material for simulation and comparison, due to its strength to withstand compressive forces in the tracheal loading conditions, as well as its biocompatibility in the host body. Using Solidworks™ assembly module, cartilage rings from the diseased portion will be removed and replaced with the designed scaffold. In this simulation study, the 5^{th} and 6^{th} cartilage rings from the top of the tracheal model were removed and replaced with the designed scaffold (FIG. 5). This is to simulate a real life scenario, whereby the entire collagen matrix of the implant has been broken down and only the scaffold is left embedded in the newly formed membranous tissues, which takes over the function of the removed cartilaginous rings. For comparison purposes in the simulation, both modular circular shaped hollow prosthesis and elliptical shaped hollow prostheses (FIG. 13) were designed using Solidworks™. Material properties of both 316L stainless steel and CNT-PDMS composite are also being input into both geometrical designs for results comparisons. The assembly is put through stretching and bending motions in COMSOL™ Multi-physics to study the stress concentrations in the different regions during daily motions of the trachea. For this invention, stretching by 10% strain (due to swallowing) and bending in the Y and Z directions (from head and heck movements) were done on both natural and augmented trachea models to study the effect of the implant on the surrounding natural membrane and cartilage rings. Results were taken from the maximum stress in the surrounding membrane and from the closest cartilage ring and tabulated in a table for comparison. From the results (FIG. 7B, FIG. 8B, FIG. 9B), CNT-PDMS composite was confirmed to be the material of choice for the scaffold due to closer similarities in stress concentrations to the native tracheal rings and membrane. Furthermore, results data also pointed out a reduction in stress concentrations when a user centric design is used rather than a modular cylindrical shape.

### Representative Manufacturing Methodology

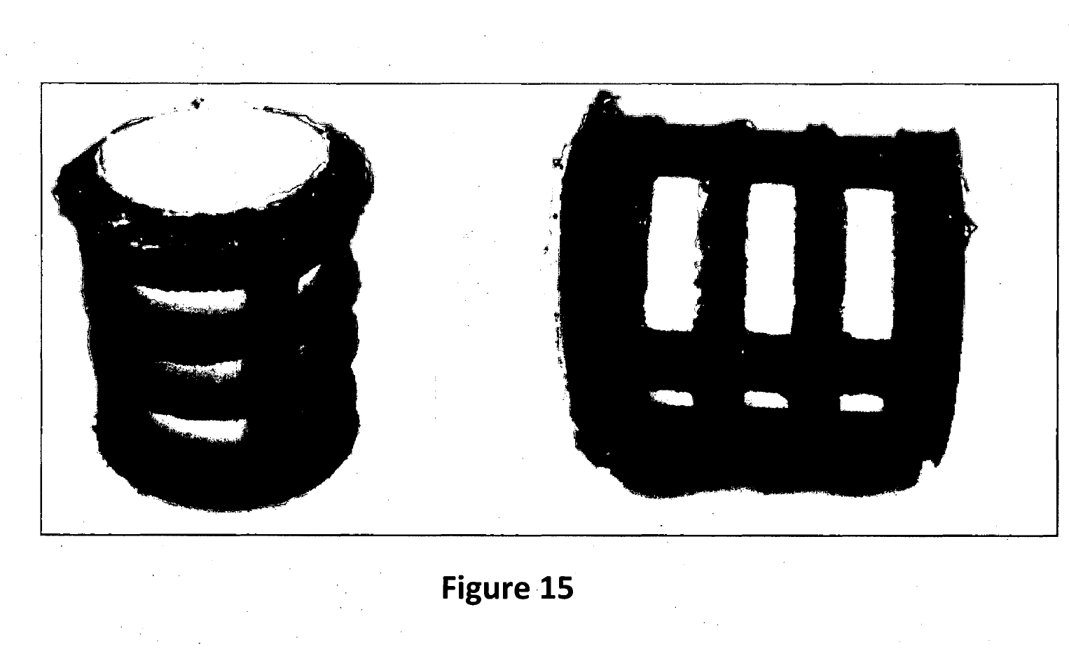

This section \relates to a methodology for the fabrication of a patient specific artificial tracheal prosthesis. The final fabricated product is illustrated in FIG. 15. The patient specific non-biodegradation skeleton is made from 3D printing of carbon nanocomposite. First, the carbon nanocomposite filament for the rapid prototyping machine is fabricated. Sylgard 184™ PDMS is mixed thoroughly in a ratio of 10:1 with its curing agent to achieve optimal strength according to product information. Carbon nanotubes are then added into the slurry at various concentrations to different PDMS portions and stirred thoroughly in order to achieve a range of mechanical properties. The mixture will then be poured into the base mold and left in a vacuum jar or desiccator for 20 minutes to remove any trapped bubbles which would affect the mechanical properties of the final product. Following which, it is left to cure at room temperature (100° C.) for 30 minutes and extruded through a die to form the filaments. Depending on the desired mechanical properties of the scaffold, the appropriate filament is selected and loaded into the 3D printer and the prosthesis skeleton is fabricated out. A multi-material electrohydrodynamic jet printer can also be used for the fabrication of the carbon nanocomposite device as it can have better control over the spatial composition of the implant. These methodologies can be applied to essentially any nanofiber-thermoplastic composite combinations used to fabricate medical devices.

For the fabrication of the Type I collagen sponge layer, a rectangular aluminum mold was machined. Type I collagen (porcine) solution is dissolved in aqueous hydrochloric acid (pH 3) to give a final concentration of 1% by weight (Yamashita 2007). This is followed by homogenizing of the solution at 8000 rpm for 15 minutes. This solution is then poured carefully into the mold cavity and the entire mold is placed into a freeze drier at −80° C. After which, the mold is placed into a vacuum oven at 140° C. for 12 hours for cross linking to occur. Upon completion, the collagen sponge is rolled and attached to the lumen of the CNT-PDMS skeleton using bioglue Coseal™. The final product should be sealed in an air tight plastic packaging and UV sterilized thoroughly.

### Representative In-Vitro Experimentation

This section provides a methodology for the in-vitro evaluation of the CNT-PDMS nanocomposite prosthesis. The main aim of the in vitro cell culturing is to verify the ability of the proposed PDMS-CNT bio-composite to support tracheal cell proliferation and differentiation. From literature, PDMS is a biocompatible material that has been extensively tested with cells. To the inventors' best knowledge, there has not been any medical implant made from a PDMS-CNT composite and thus this experiment serves to test the viability of such a combination for supporting cell growth. Porcine tracheas were obtained fresh from the local slaughterhouse. The pigs that were slaughtered were weighed and determined to be approximately 45 kg and healthy. Briefly, the tracheas were immersed into cold Hanks solution (Sigma, St. Louis, Mo.) to maintain the tissue freshness until it was ready for dissection of the mucosa layer. The epithelial mucosa was carefully removed from the tracheas and washed 5 times using M199 (Bio-Source International, Camarillo, Calif.) with antibiotics. After which, the tissues were sliced into small pieces and incubated at 4° C. overnight in M199 supplemented with 1× of penicillin/streptomycin (Gibco, Grand Island, N.Y.) and 0.6 mg/ml type IV protease (Sigma, St. Louis, Mo.). Clusters of epithelial cells were harvested the following day by gently agitating the pieces of the sliced mucosa in a Petri dish containing M199 with 10% FCS (ATCC, Manassas, Va.). The cells were then washed five times with M199 supplement with 1× of penicillin/streptomycin mix and re-suspended in medium BEGM (Lonza, Walkersville, Md.) containing 5% FCS and 10⁻⁷M RA. The ingredients used in the BEGM include: epidermal growth factor (0.5 ng/ml h_EGF), insulin (5 μg/ml), hydrocortisone (0.5 μg/ml), transferrin (10 μg/ml), epinephrine (0.5 μg/ml), triiodothyronine (6.5 ng/ml), bovine pituitary extract (60 μg/ml), gentamicin (50 μg/ml), cholera toxin (10 ng/ml), retinoic acid (0.1 ng/ml), amphotericin (50 ng/ml) and 0.8% penicillin-streptomycin. The cells were then stored under cryopreservation until they were needed. The scaffold being tested is the proposed PDMS-CNT bio-composite. Scaffold pieces that were 1 by 1 cm in dimension and 0.2 cm thick were prepared. The surface of the composite was treated with collagen to enhance cell adhesion before seeding. 50 mg of Purified Type I Collagen (Symatese, Chaponost, France) was dissolved in 100 mL of 0.2% glacial acetic acid and homogenized at 5000 rpm for 10 minutes under cooling from ice. The solution was then further diluted 1:5 with double distilled water. Each scaffold was coated with around 50 uL of the collagen and incubated at room temperature overnight. The excess solution were then removed from the scaffold and air-dried under sterile conditions. Prior to seeding, the batch of bio-composites were subjected to UV sterilization and rinsed with PBS containing antibiotics. One scaffold was placed into each well of the 12-well plates (Corning, USA) and 2 ml of BEGM growth media was added in and pre-incubated at 37° C. for 2 hours and then aspirated. Approximately 10,000 cells (in 100 uL growth medium) were seeded onto each scaffold and incubated in at 37° C. with 8% CO₂ and 95% humidity for 4 hours for attachment to take place. After which, the scaffolds were transferred in new well plates and fresh 2 ml BEGM growth medium were added in each well before being placed in the incubator. The old well plates were inspected under the microscope for any residual cells to ascertain that the tracheal cells have attached to the scaffold surface. The culture media was changed every 2 days. On the 2^{nd}, 5^{th} and 7^{th} day, samples were taken out for confocal microscope, SEM and assessment.

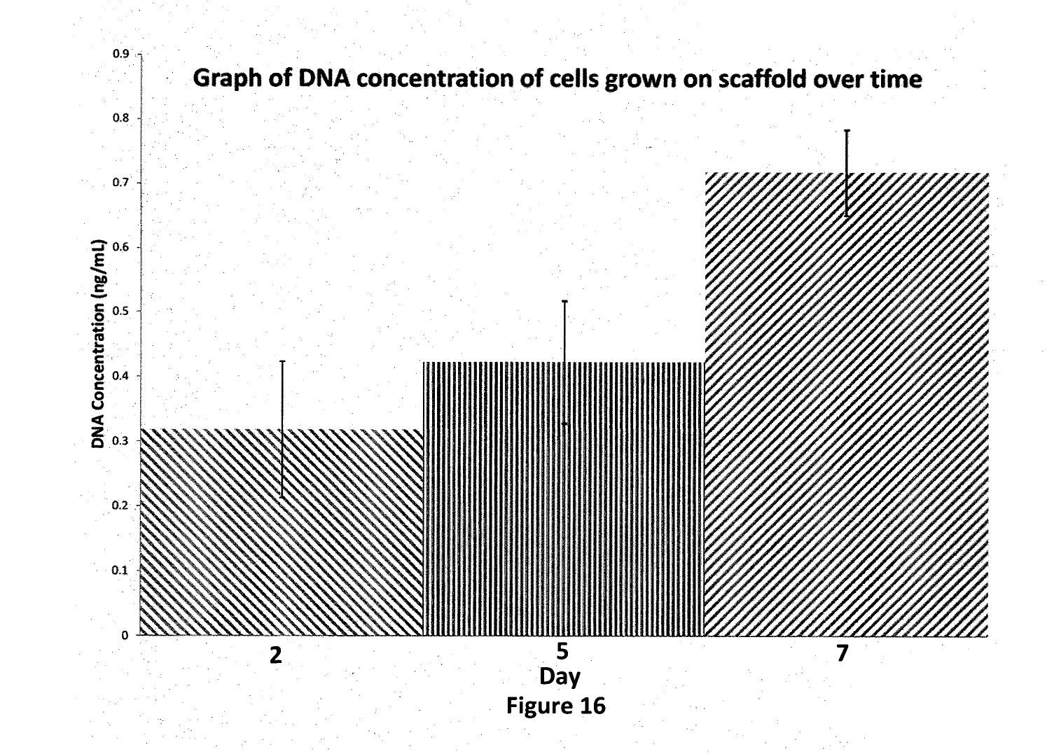

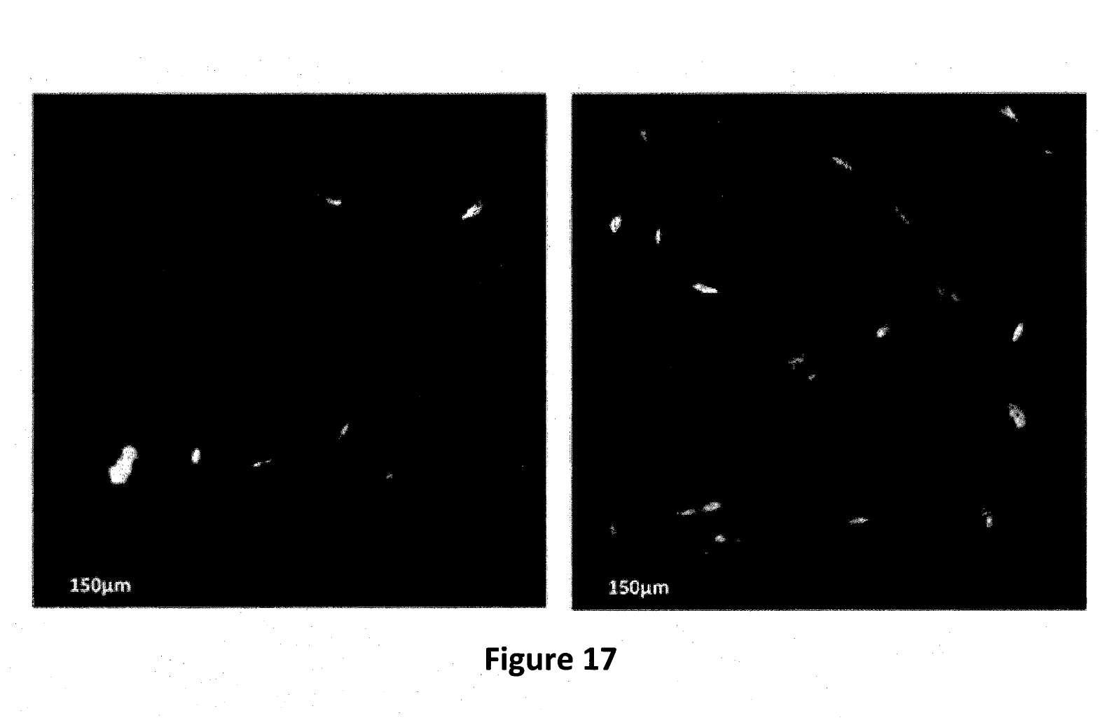

From the microplate readings of the PicoGreen® stained samples (FIG. 16), an increased in the DNA concentration was observed over the period of 7 days. The readings are obtained from a sample size of 3 per time point. In the images captured by the fluorescence microscope (FIG. 17) for LIVE/DEAD® staining of cells, greater amounts of live cells were observed than dead cells in both time point of 2^{nd} and 7^{th} day. The density of live cells also seems to be greater on the 7^{th} day compared to the 2^{nd} day. In addition, confocal images seemed to show the formation of cell adhesion to each other, hence indicating the possibility of a suitable growth environment on the scaffold. These results indicate that the PDMS-CNT bio-composite is able to provide a biocompatible and conducive environment for the proliferation of the tracheal cells.

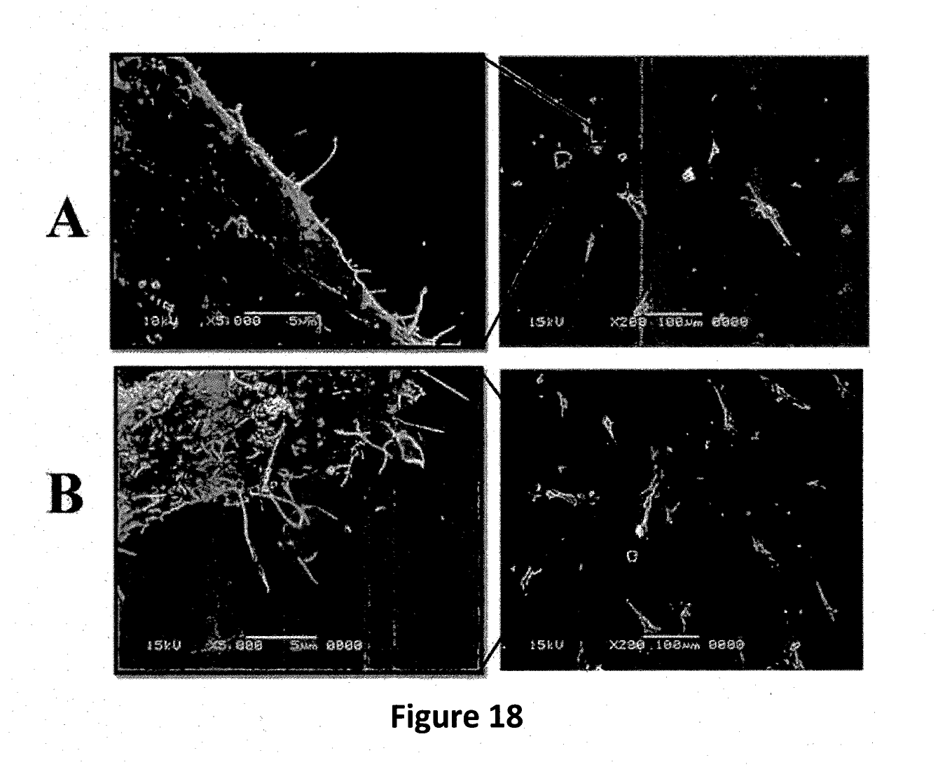

SEM images of the samples (FIG. 18) shows that the cell surfaces are almost devoid of cilia on day 2 of the cell culturing. By day 5, it is observed that a substantial density of cilia was observed on the surface of the tracheal epithelial cells. The ciliated structures found on the surface of cells are comparable to those in literature (Chopra, Kern et al. 1992; Ziegelaar, Aigner et al. 2002; Mao, Wang et al. 2009). This indicates that the proposed composite scaffold was able to allow differentiation of the trachea cells to become ciliated, hence their functionality was ensured. A greater tracheal cell density was also observed in the SEM images on day 5, this is consistent with the results from LIVE/DEAD® staining and Picogreen® staining which all indicates the suitability of PDMS-CNT nanofibers composite surfaces to host cell proliferation.

### Representative Deployment Methodology

This section provides a deployment methodology of the artificial trachea, considered in a porcine model. The aim of this in vivo animal experiment is to assess the ease of implantation of the prosthesis and the effectiveness of the replacement graft. Briefly, a pre-operation CT scans were conducted on 5 healthy female pigs, weighing around 60 kg, to obtain the cross sectional image of the tracheas, following which the tracheal prostheses were fabricated based on the dimensions of the native tissue. The animals were then anaesthetized and placed in a supine position on the operating table. Incisions were made on the tracheas and 3-4 tracheal rings were removed. The prosthesis was then soaked in the pig's blood to render it air tight before it was sutured to the resected ends of the trachea. The pig was then sutured up and antibiotic ointment was applied on the wound to prevent infection. Antibiotics were prescribed and standard post-operative care regimes were undertaken according to IACUC protocols. The pigs were kept alive for 3 weeks and sacrificed. The prosthesis and the surrounding tissues were carefully harvested for histological studies.

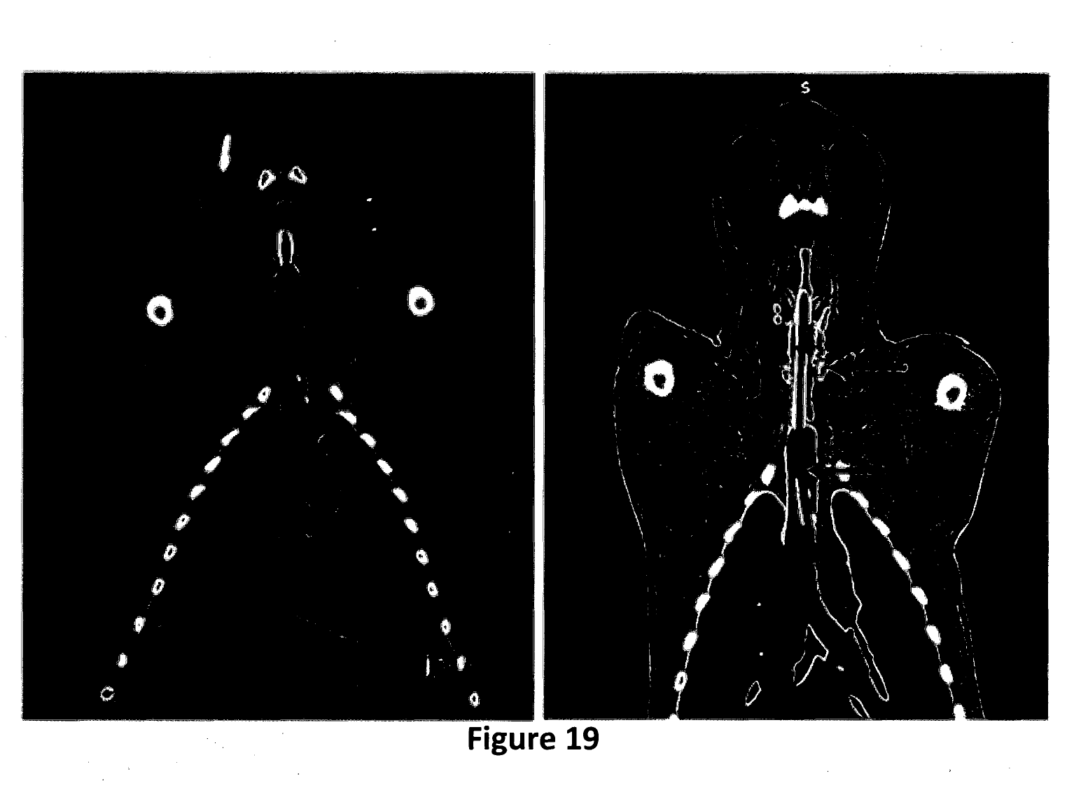

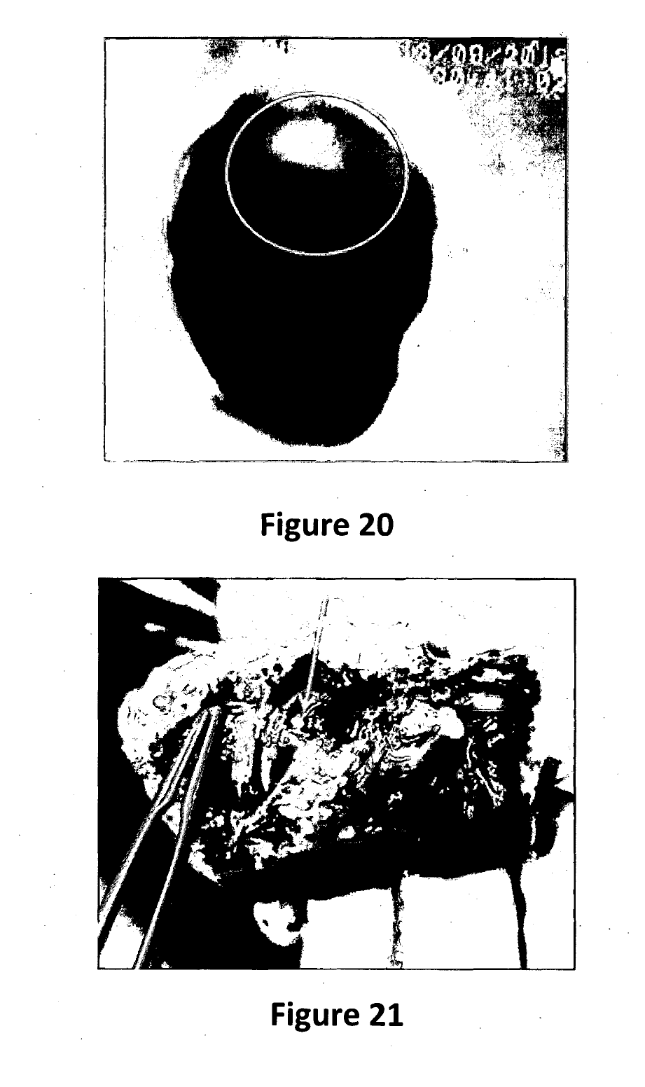

Preoperation and post-operation CT scans of the pig in FIG. 19 shows the graft maintaining airway patency. During the 2nd week, the pigs were put under general anaesthesia and a flexible endoscope was introduced into the trachea to observe the lumen. It was observed that the inner lumen of the prosthesis has been covered with a layer of epithelium (FIG. 20). At the point of sacrifice after three weeks and the harvesting of the tissue-scaffold, it was observed that there was substantial tissue in-growth into the prosthesis (FIG. 21). The scaffold has shown to be able to maintain airway patency with no particular breathing difficulties observed from the pig.

Tracheal prosthesis embodiments in accordance with the present disclosure solve the problem of maintaining sufficient mechanical strength to withstand surrounding pressure, yet have almost similar flexibility as the natural tracheal rings which allows restoration back to an original cross section after compression. Both the predominantly Type I collagen and the VEGF drug provide a conducive environment for the in-growth of blood vessels and cells and accelerates their proliferation. Finally, tracheal prostheses in accordance with embodiments of the present disclosure are purely synthetic and biocompatible with the human body, thus requiring no seeding of autologous cells or consumption of immune-suppressant after implantation. In view of the foregoing description, tracheal prosthesis embodiments in accordance with the present disclosure can overcome limitations of existing tracheal prostheses.

### Voice Prosthesis

This invention presents a methodology and material for the design, simulation, fabrication and testing of a voice prosthesis, which can be a patient specific voice prostheses. One embodiment of the present disclosure overcomes the fistula shape and size specificity of each patient by utilizing one or more images captured of the patient's fistula and extracting the dimensions from its area to recreate patient specific voice prostheses. It presents a novel magnetic ball bearing as a one way valve construct in the voice prosthesis to prevent entry of food and water from the esophagus while allowing air from the trachea to flow through to enable speech. This invention also describes the usage of carbon nanotubes—polymer composite as the material for the prosthesis which gives it better mechanical properties. Another embodiment of the present disclosure overcomes the fistula shape and size differences between patients by utilizing the image(s) captured of the patients to recreate patient specific voice prostheses. A multi-layered prosthesis skin can also adapt to compensate for minor changes in the patient's fistula over time. A novel magnetic ball bearing valve in the voice prosthesis has high backflow resistance to prevent leakages, while maintaining low forward flow resistance for ease of speech. The ball bearing also allows for variation in pitch and the creation of a more natural sounding speech. Also described is the usage of the CNT-PDMS nanocomposite as the prosthesis material to mimic the mechanical properties of the surrounding tissues while having potential in creating a more natural speaking tone.

### Representative Process Flow

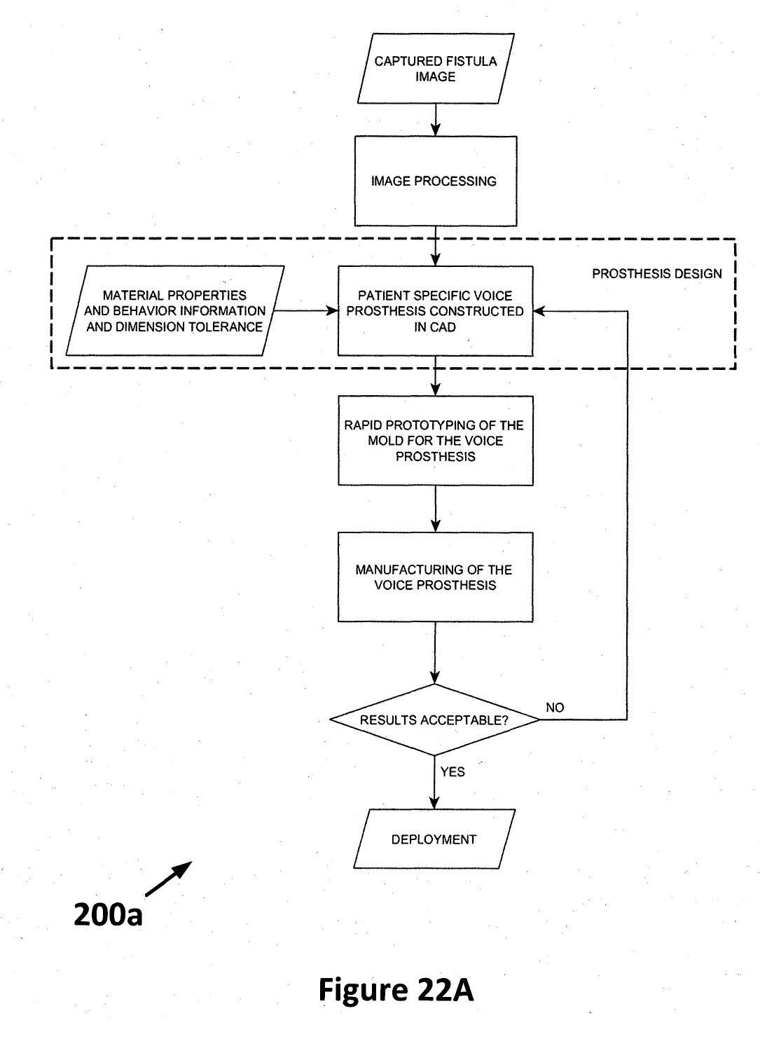

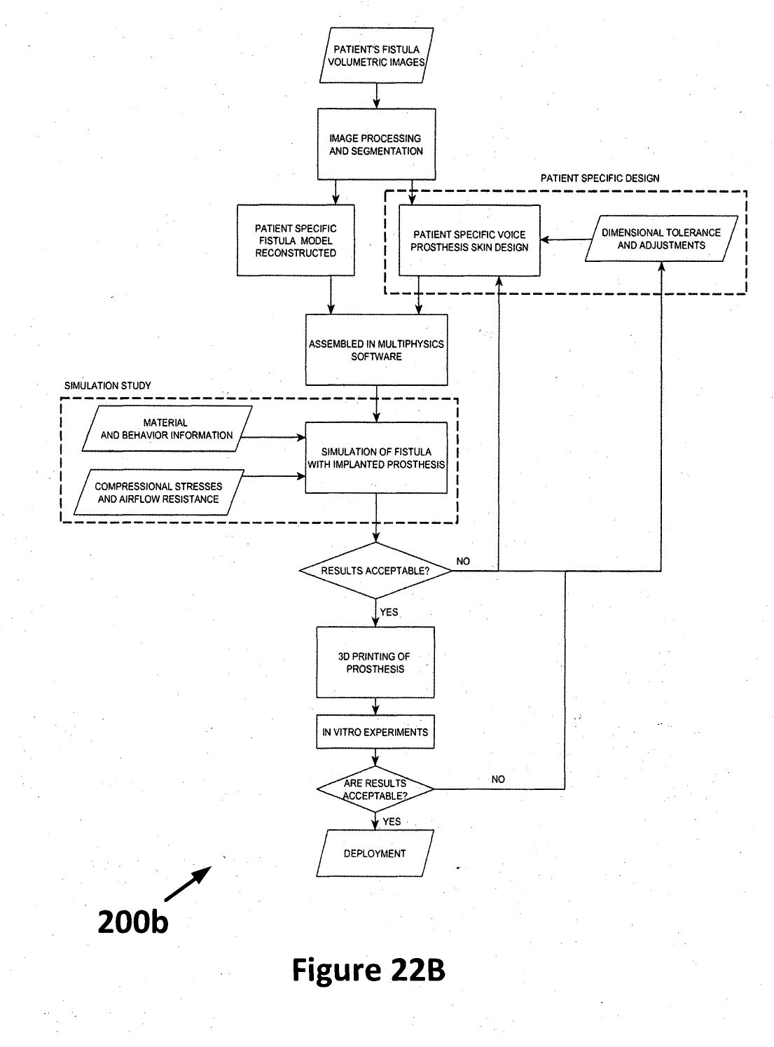

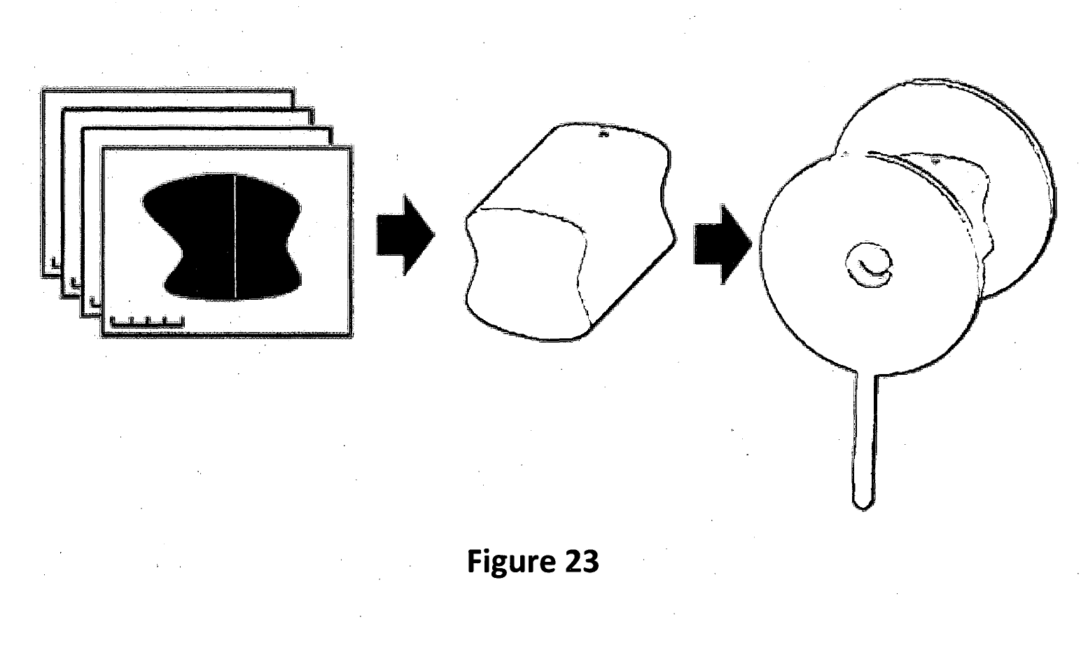

A schematic diagram for an invention flow process for one embodiment of the present disclosure is shown in FIG. 22A. Basically, after puncture, an image of the fistula is captured which includes or shows its size and dimensions. The image is run through image processing software and the exact, nearly exact, or approximately exact geometrical shape and size of the cross section of the fistula is extracted and imported into CAD software. From this cross section and the measured fistula depth, a customized patient specific voice prosthesis (FIG. 23) can be created. Tolerance for the size will be factored into the design to ensure that the prosthesis is fitted securely into the fistula even if minor enlargement of the hole occurs. Based on the designed prosthesis, a mold for it is created in CAD software, and rapid prototyped out for the fabrication of the voice prosthesis. PDMS pre-polymer and carbon nanotubes are mixed thoroughly together to ensure a homogenous mixture before being poured into the mold. The mold is then left in a vacuum oven for 1 hour to remove any bubbles within, followed by curing at 70 degrees Celcius for at least 4 hours. The resultant molded prosthesis is then inspected for any flaws before the magnetic ocular seating and magnetic ball bearing are added to create the one way valve.

### Representative Design and Materials for Voice Prosthesis

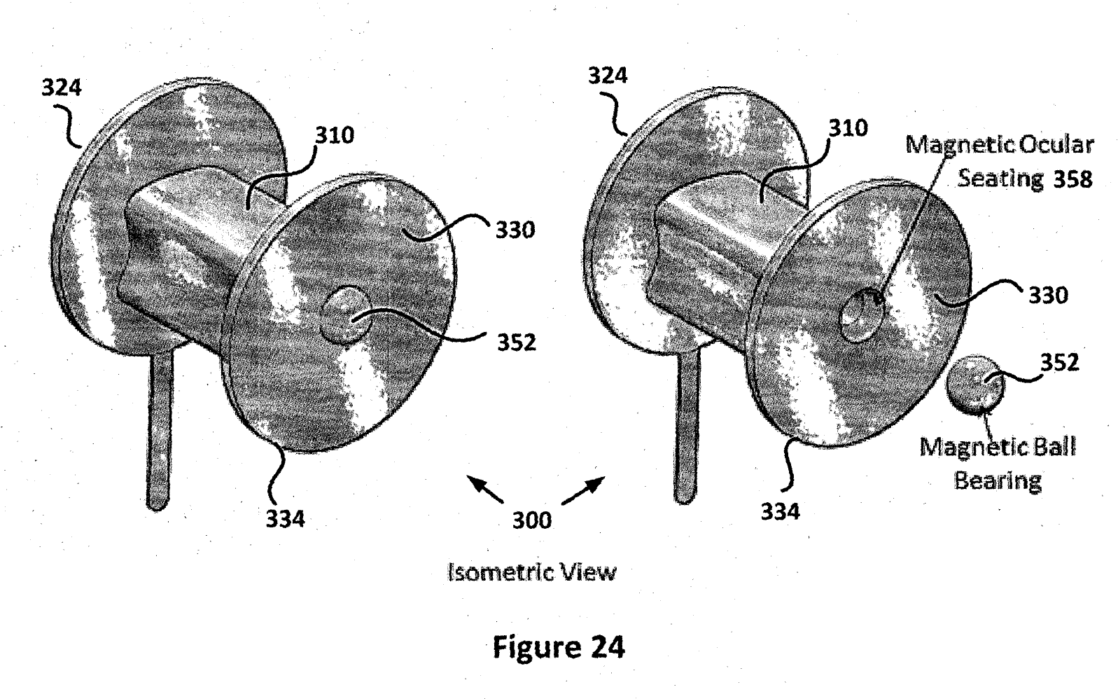

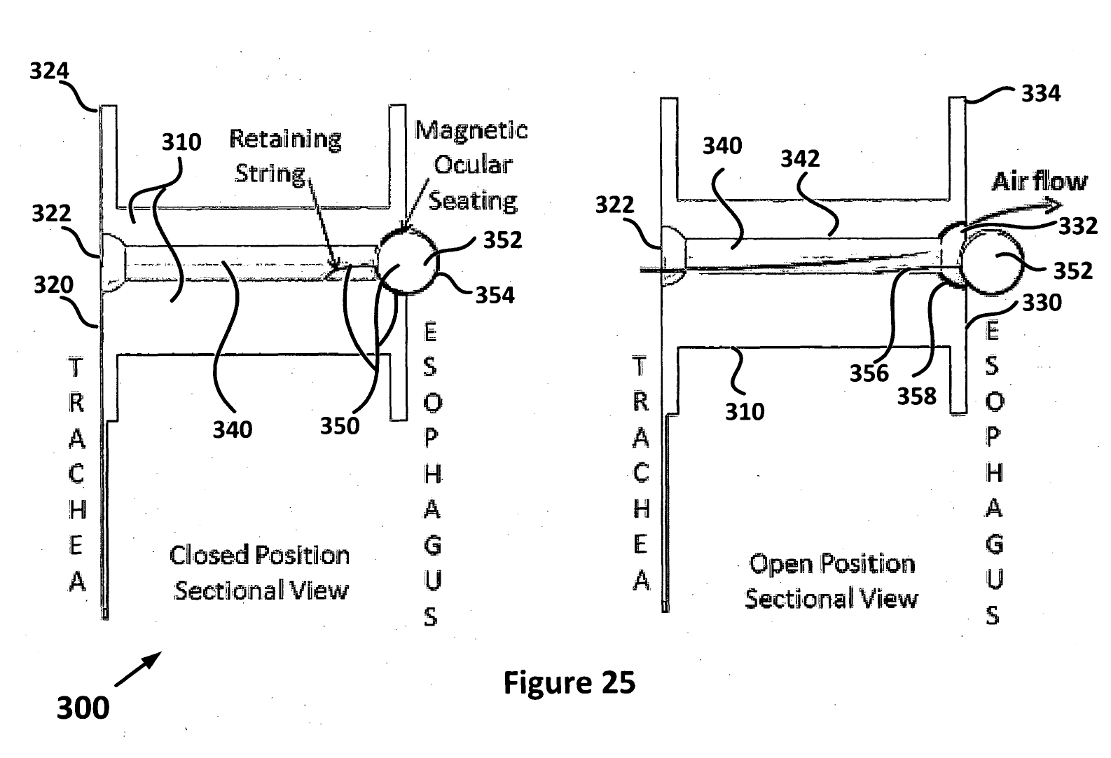

This section describes the design and materials needed to create the patient specific voice prosthesis. An isometric view of the overall voice prosthesis can be found in FIG. 24. Basically, the voice prosthesis includes or is a bio-composite body made of carbon nanotubes and PDMS, with a magnetic ocular seating for the ball bearing at the esophagus end and a magnetic ball bearing held by a restraining string to the body of the prosthesis. The dimensions of the body of the prosthesis like its length and geometrical cross sectional shape are determined by the image(s) captured form the patient's fistula. One can understand the working mechanism of the ball bearing valve in FIG. 25. During normal breathing, the magnetic ball bearing is attracted to the magnetic ocular seating and seals the passageway, hence preventing any food or water from entering the trachea from the esophagus. During speech, the stoma is covered and air is channelled into the prosthesis, hence forcing open the magnetic ball bearing, which allows air to enter the esophagus into the vibrating segment for speech production. The ball bearing is held securely to the prosthesis via a restraining string to prevent it from falling into the esophagus.

A schematic diagram for the invention flow process for another embodiment of the present disclosure is shown in FIG. 22B. Basically, after the puncture, volumetric images of the patient's fistula are captured via CT or MRI. The images are processed and used to model the patient specific voice prosthesis (FIG. 23). Tolerance for the size and adjustments will be factored into the design to ensure that the prosthesis can be fitted securely into the fistula even if minor enlargement of the hole occurs. A patient specific fistula model is also recreated from the images; and simulation studies can be performed to ensure that the design is optimized. Once deemed satisfactory, the patient specific skin of the prosthesis will be rapid prototyped out. The inner rigid core made from PVC is machined and the magnetic ball bearing valve is installed. After which, the CNT-PDMS skin and the PVC core are assembled. The prosthesis is then subjected to in-vitro flow and leak tests before being deployed. Deployment can involve testing in a porcine model for in-vivo evaluation.

### Representative Design and Materials for Voice Prosthesis

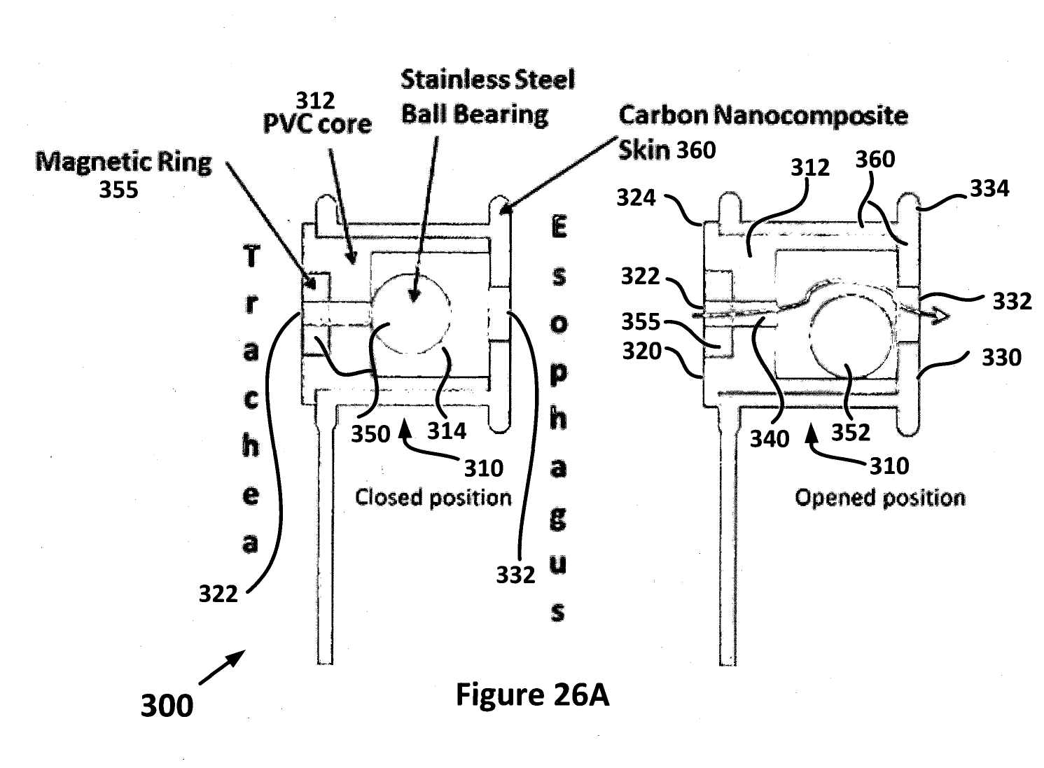

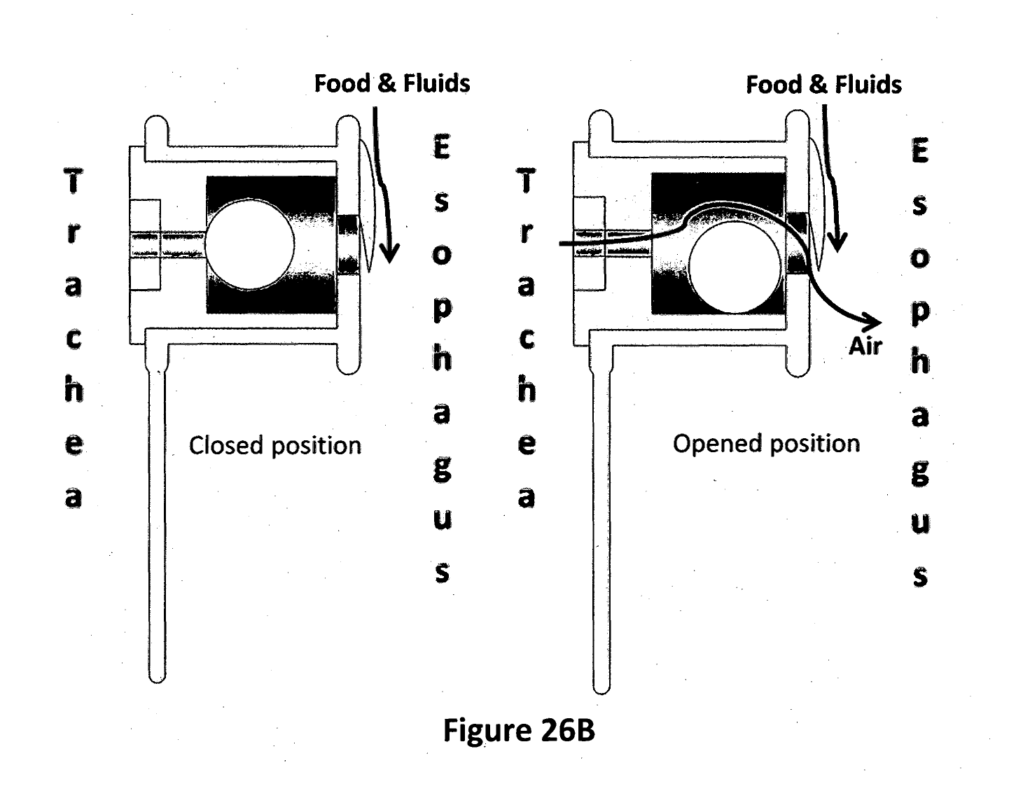

This section describes the design and materials for creating the patient specific voice prosthesis. A sectional view of the proposed voice prosthesis can be found in FIG. 26A. Basically, the voice prosthesis includes two main parts: a patient specific carbon nanocomposite skin filled with sponge or silicone gel, and a PVC core rigid body that houses the magnetic ball bearing valve. The small modular design of the rigid PVC core allows it to maintain airway patency under compression stresses and it also allows for different dimensions of carbon nanocomposite skin to be easily sheath over it for better fit. One can find the working mechanism of the ball bearing valve in FIG. 26A. During normal breathing, the magnetic ball bearing is attracted to the magnetic ocular seating and seals the passageway, hence preventing any food or water from entering the trachea from the esophagus. During speech, the stoma is covered and air is channel into the prosthesis, hence forcing open the magnetic ball bearing, which allows air to enter the esophagus into the vibrating segment for speech production. In some embodiments of the present disclosure, an umbrella can be added over the entrance that helps to guide food and fluid over it downwards (FIG. 26B)

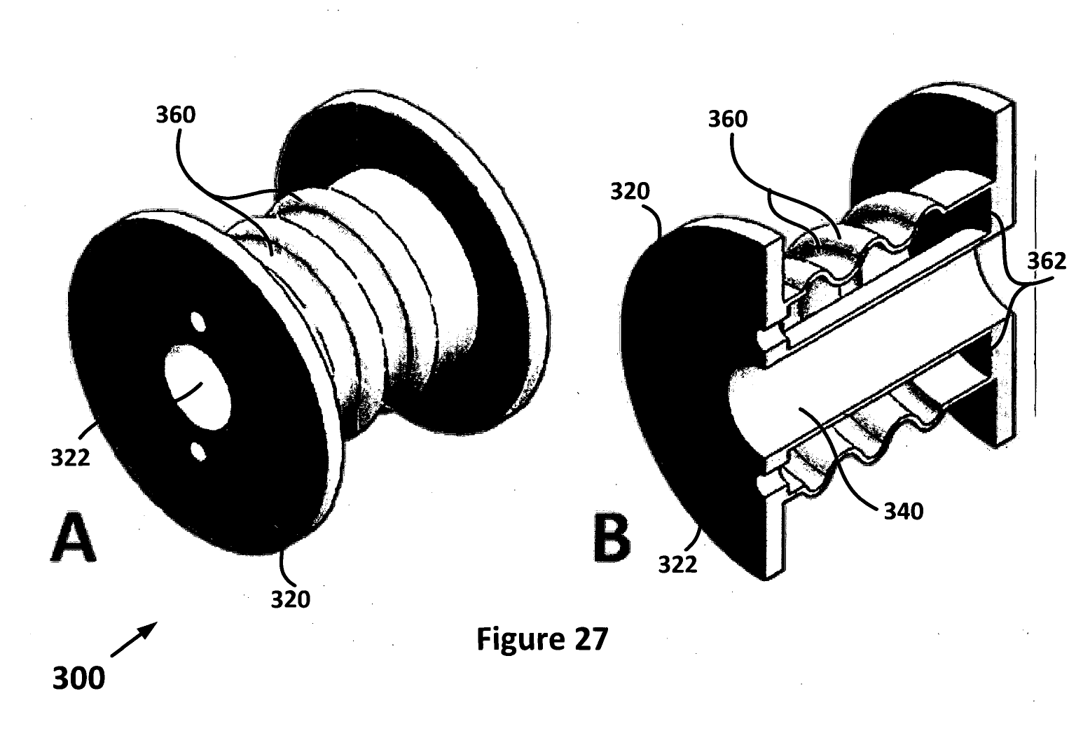

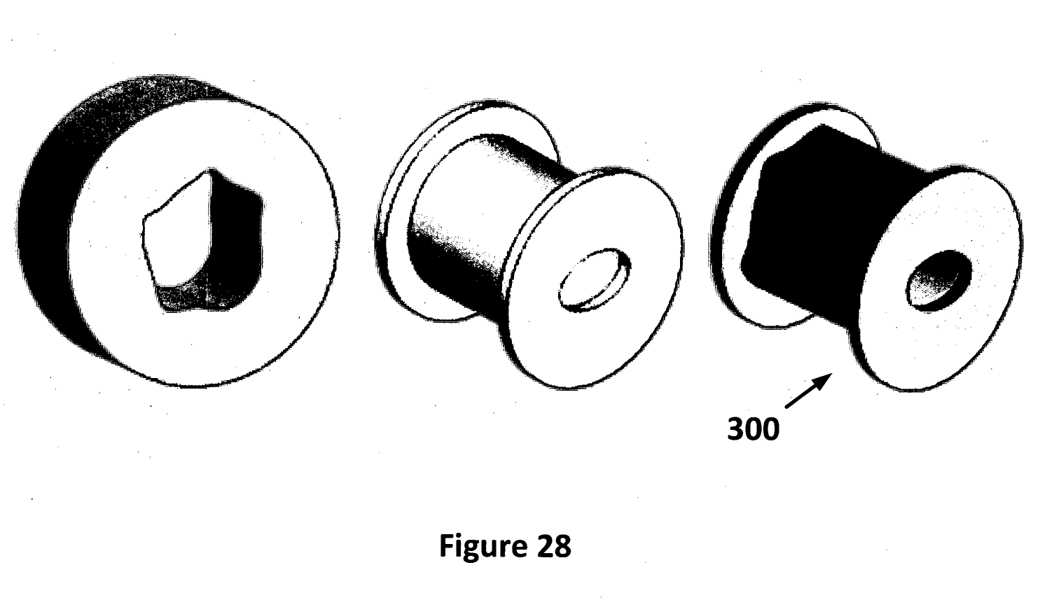

The carbon nanocomposite skin acts as the interface between the rigid prosthesis core and the surrounding fistula tissues. The skin may be completely made of carbon nanocomposite, or a cavity can be incorporated into its design (FIG. 27) whereby deformable material like PVC sponge or silicone gel can be filled in. The carbon nanocomposite has material properties that are similar to the surrounding tissues that help to reduce tissue irritation and stress levels. Furthermore, a patient specific design of the skin helps to evenly spread out the stress concentration around adjacent tissues. The spongy or viscous liquid contained within the skin allows for adaptations to minor changes to the fistula dimensions without the need to change to a new skin. This will result in cost savings, and prevents discomfort to the patient.

### Representative Simulation Study Methodology

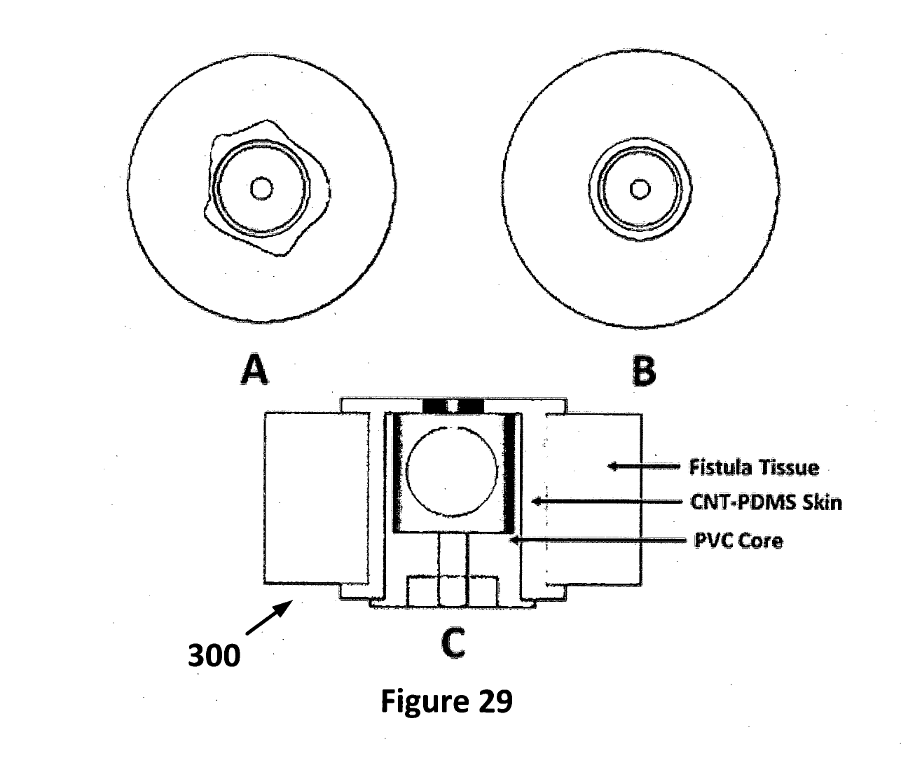

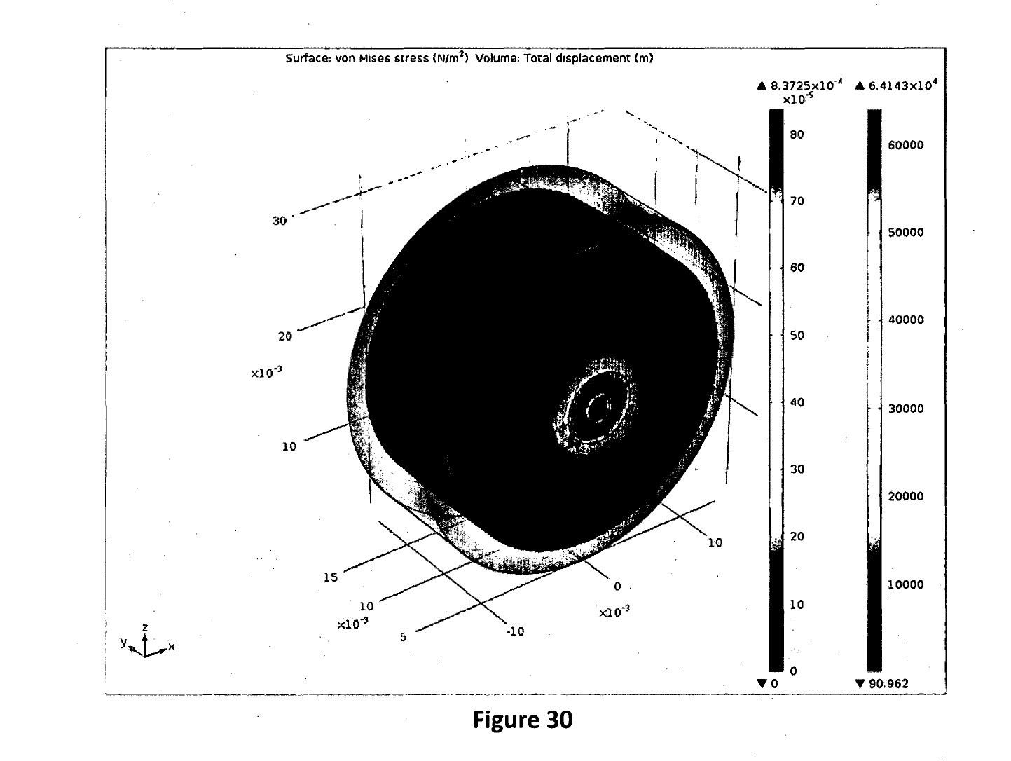

This section describes details for the in-silico evaluation of the voice prosthesis to optimize its flow resistance and patient specific dimensions. Two different computational simulations were performed for performance evaluation as part of the CAD process. In the first simulation, a stress comparison and analysis was done for both the patient specific voice prosthesis and a regular cylindrical prosthesis designs (FIG. 28). The prosthesis and fistula models were assembled in a multi-physics program (FIG. 29) and their respective material parameters were assigned. Recent research has utilized the Holzapfel strain energy of two orthogonal families of fibres for the membranous fistula wall. However, this was invalid for a 3D dynamic model due incompressibility issues. Thus, the fistula soft tissue was modelled as an orthogonal behavioural material with different material properties in all three principle axis according to the values from Sarma et al. Inwards radial stresses ranging from 1.5 kPa to 9 kPa were applied on the tissue to simulate real life tissue compression by the inserted voice prosthesis and the von Mises stress distribution in the tissue was examined (FIG. 30). The second simulation study involves computing the pressure change against airflow rate of the prostheses using multi-physics flow module software. The purpose is to analyze the airflow resistance of the proposed voice prosthesis designs. Briefly, CAD models of the proposed magnetic ball bearing valve of inner minimum diameters of 1 mm, 2 mm, 3 mm, 4 mm and 5 mm were prepared. The first pressure probe was positioned at the entrance of the tracheal side while the second one at the oesophageal side. Air flow rates within the human speaking range, 50 mL/s to 300 mL/s, were simulated in the prosthesis models and the pressure readings were recorded.

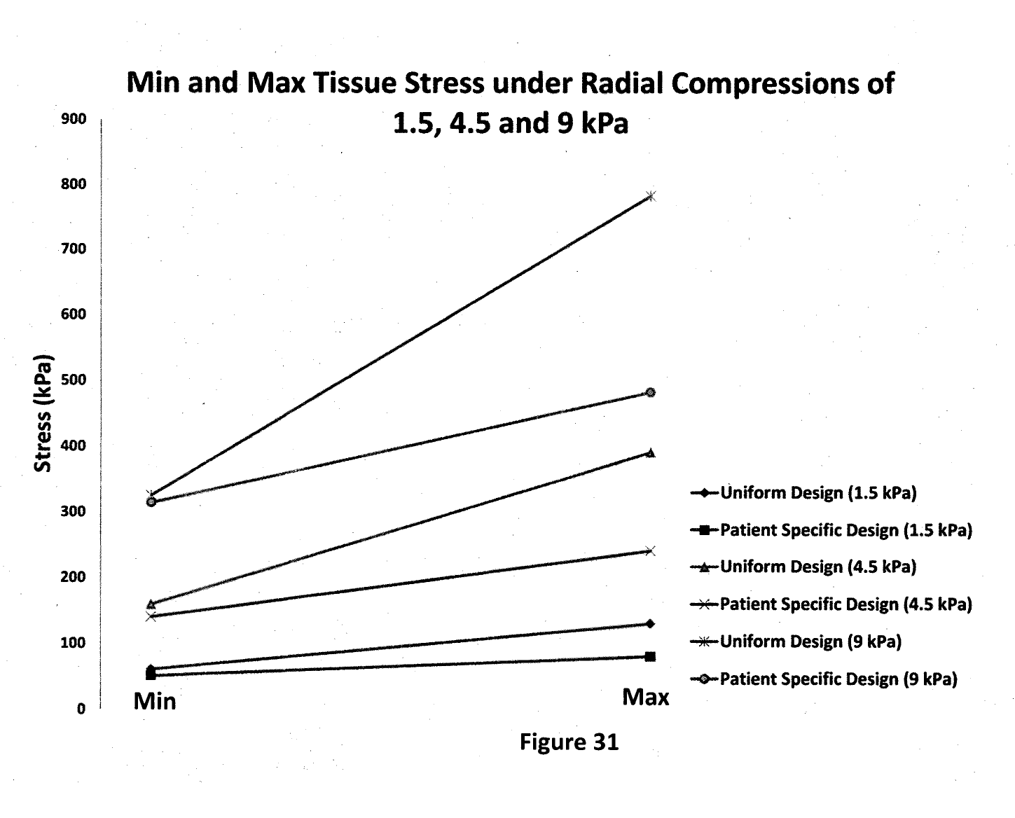

The von Mises stress values in the fistula tissue were recorded for both uniform design and patient specific design voice prostheses under various radial compressions in FIG. 31. From the graph, it can be seen that a patient specific design will result in lower minimum and maximum stress values in the surrounding tissues for different radial compression values. This may be attributed to a better fit of the prosthesis in the fistula hence resulting in a more even stress distribution around the tissue. The matching geometry between prosthesis and the fistula also led to greater reduction in maximum stress as increasing radial compression is exerted.

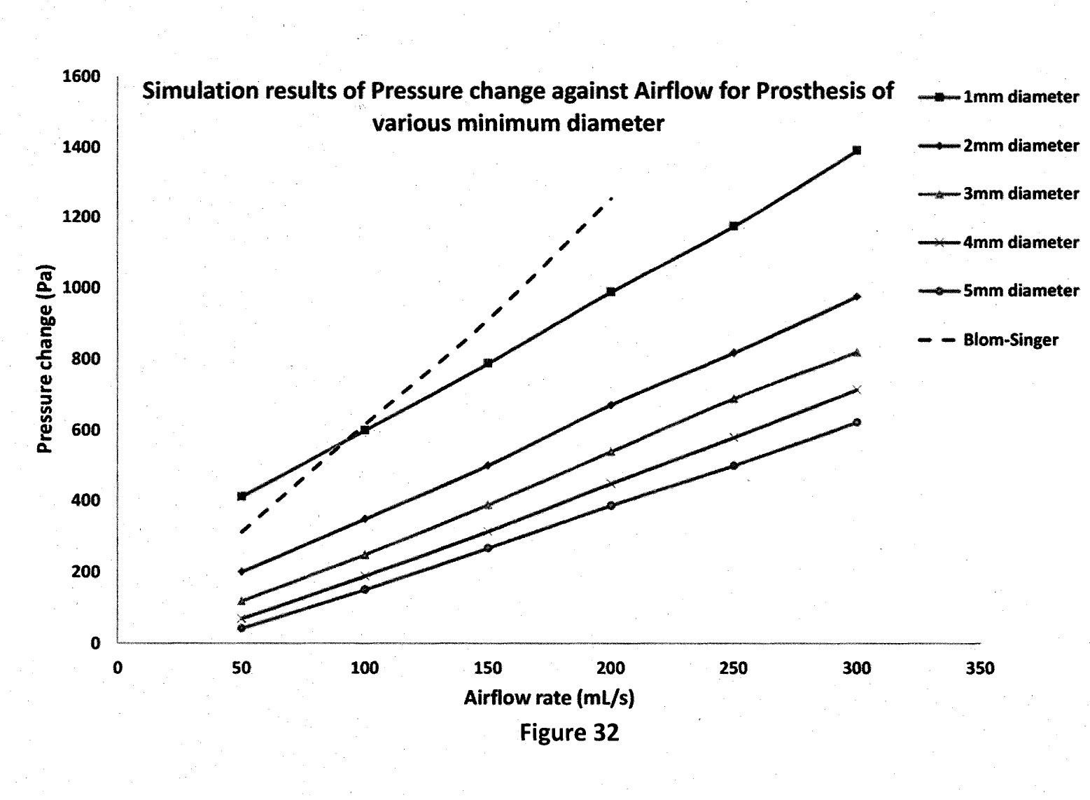

The results from the second simulation study on the air flow resistance for voice prostheses of different inner minimum diameter was tabulated and plotted (FIG. 32). The resistance of the prosthesis at a particular airflow rate is the ratio of pressure change to airflow rate. Smaller diameter prostheses are desirable due to reduced trauma on the surrounding tissues during insertion, but flow resistance increases with decreasing diameter. Therefore the purpose of this study is to determine the smallest diameter for the design, while ensuring that flow resistance is kept reasonably low. The Blom-Singer voice prosthesis was selected as the bench mark for design. From the results, optimal inner diameter would be 2 mm as it is the smallest diameter for which airflow resistance is less than the Blom-Singer prosthesis. Through this process of computer aided design and analysis, the design of the medical device can be optimized easily and efficiently.

### Representative Manufacturing Methodology

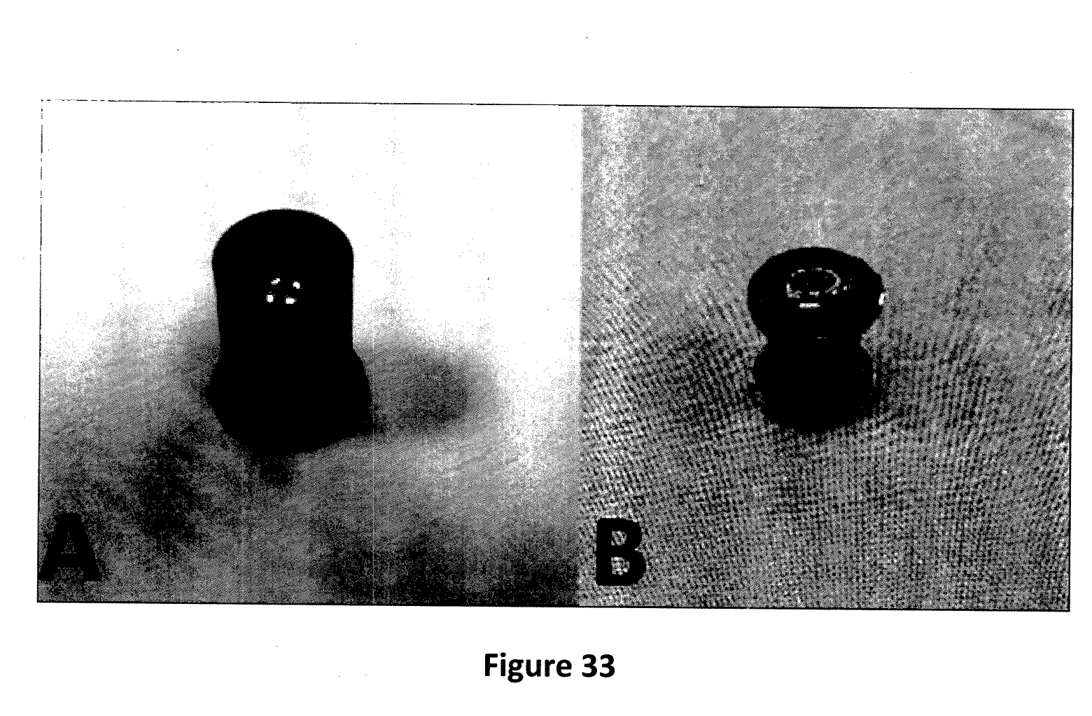

The CNT-PDMS skin was designed according to the patient specific dimensions and fabricated via 3D printing. Fabrication of the CNT-PDMS filaments is similar to the method described above with respect to the artificial trachea. Alternatively, the patient specific CNT-PDMS skin can be printed using a multi-material electrohydrodynamic jet printer, which can print nano-particles and materials can vary the spatial compositions of the nanocomposites. Fabrication of the fixed dimension rigid PVC core was done through precision machining of a piece of PVC cylinder. Magnetic rings and ball bearings (Misumi, Singapore) were then mounted into the rigid core. The final products are shown in FIG. 33.

### Representative In-Vitro Experimentation

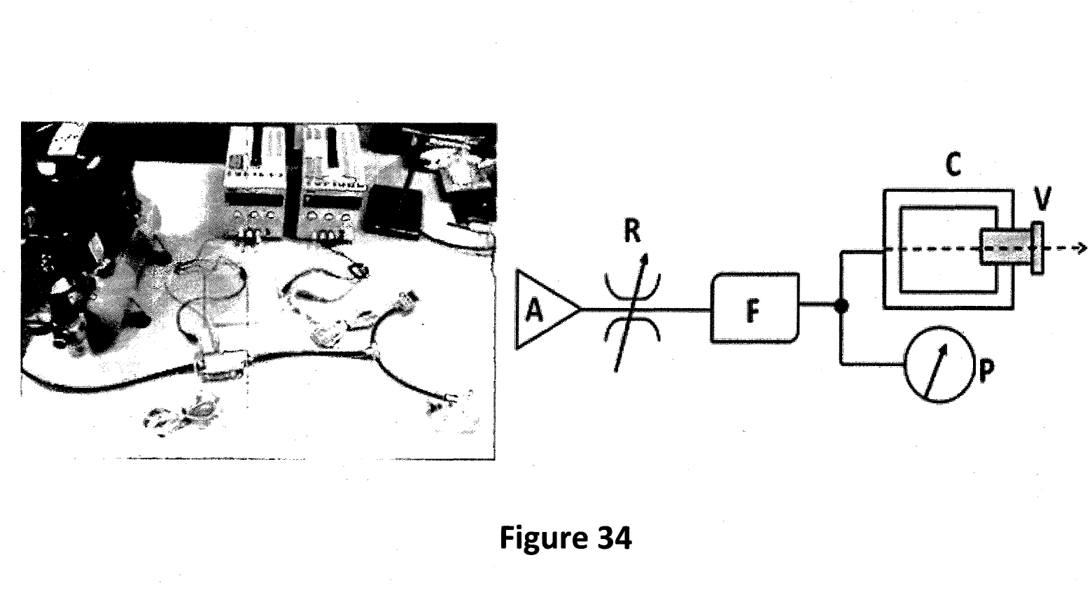

The purpose of this in-vitro experiment is to study the forward flow characteristics of the developed magnetic ball bearing voice prosthesis and the reverse flow characteristics. The forward flow characteristic is the air flow resistance through the prosthesis and it determines the ease of speech for patients. The reverse flow characteristic is the relationship between the amount of leakage through the prosthesis in the reverse direction with increasing pressure from the esophageal side. A specially constructed testing apparatus was prepared for measurement of forward flow characteristics (FIG. 34). Air supply was provided by a motorized air pump with an onboard variable flow valve, R, which could vary the amount of air flow. F represents the electronic flow meter (PFMB7201S-C8-A-M, SMC Singapore) which measures and displays the rate of air flow through it. A specially constructed acrylic pressure chamber, C, which is leak proof and houses the prosthesis, V, was fabricated. P is the pressure transducer (ISE40A-C6-X-M, SMC Singapore) that is connected parallel to the pressure chamber and effectively measures the air pressure in the chamber. The pressure change across the prosthesis can be determined by subtracting atmospheric pressure from the measured pressure. Flow rates between 50 mL/s to 300 mL/s were used, which were within the normal range of human air flow recorded during speech. The air flow resistance results were then tabulated and compared with other voice prostheses from literature.

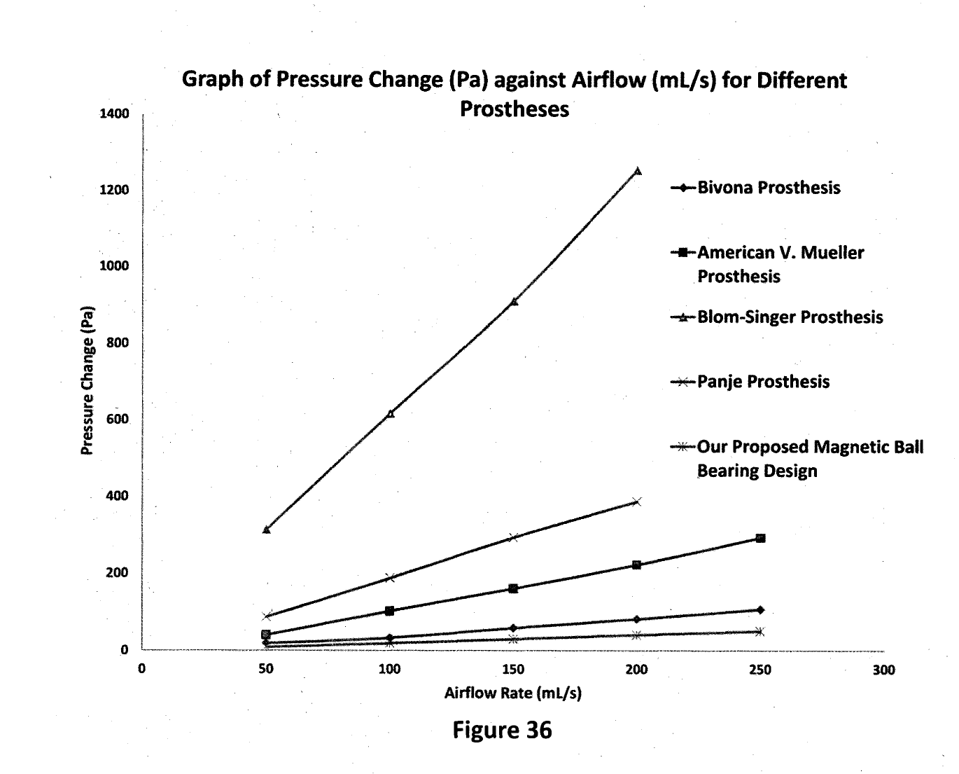

The forward flow resistance of the prosthesis is the gradient of the pressure change—flow rate graph. It is observed (FIG. 36) that the developed voice prosthesis has a lower gradient amongst the other prostheses presented and therefore a lower forward flow resistance. A low forward flow resistance implies ease of speaking in patients since high resistance would mean more effort in speaking.

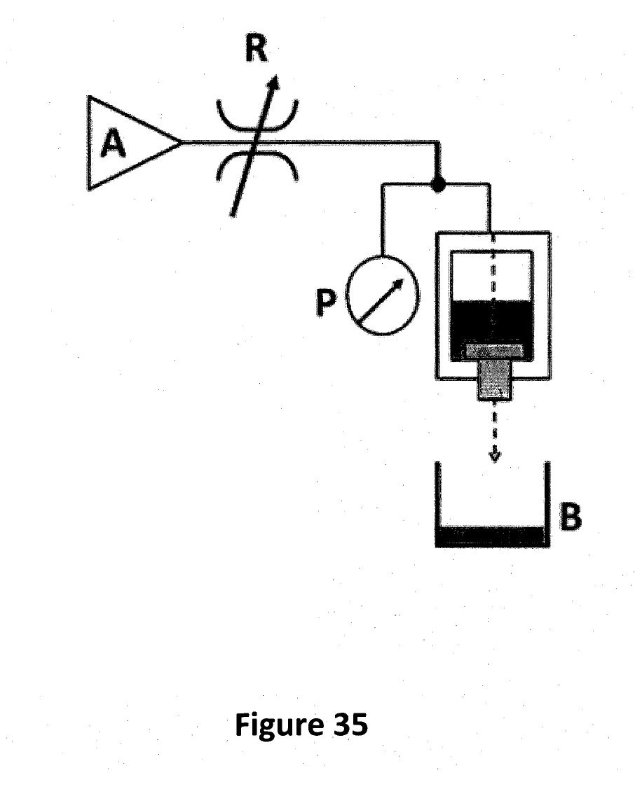

An experimental setup was designed and prepared to measure the reverse flow characteristics of the prosthesis (FIG. 35). It is paramount to assess the reverse resistance as it determines the tendency of fluid to leak from the esophagus to the trachea through the prosthesis, which could result in pneumonia. The esophageal part of the valve was subjected to dynamic pressures of 10 kPa, 20 kPa and 30 kPa for 30 seconds each before measuring the weight of water that leaks through the valve into the beaker.

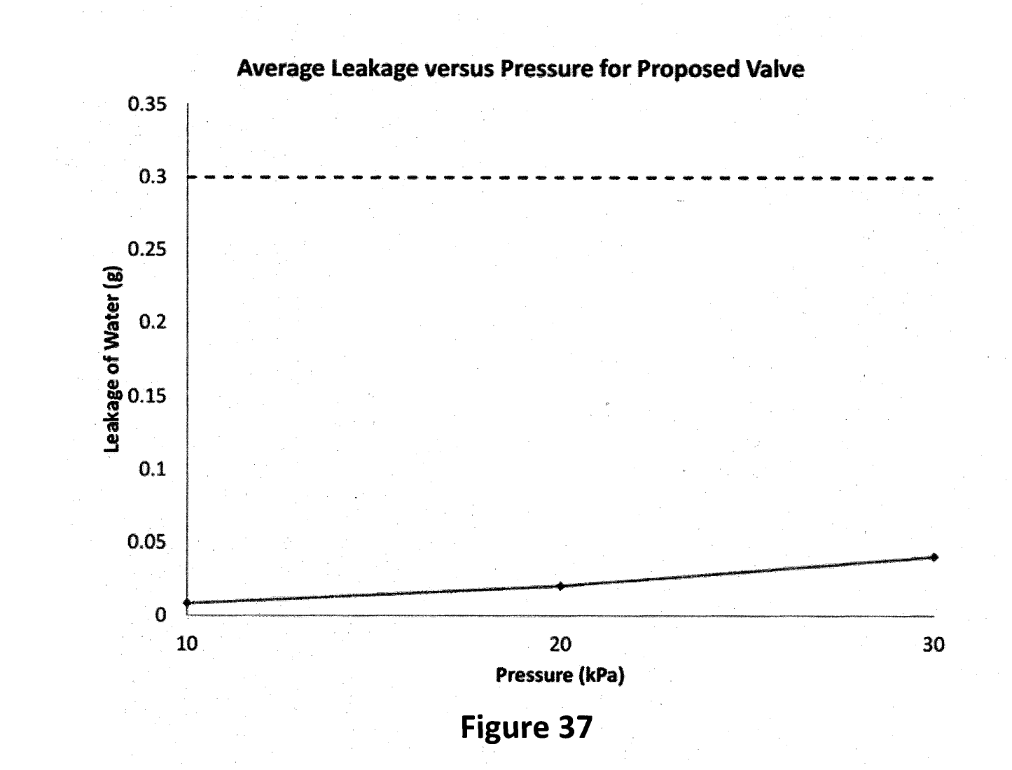

In a second experiment which determines the reverse air flow characteristics, the average water leakage was collected, weighed and tabulated into a graph (FIG. 37). It was established in literature that the average person can cough out about 0.3 g of fluid from the airway and this is denoted by the dotted horizontal line on the graph. The leakages through the proposed prosthesis throughout all 3 pressure values are less than 5% of the critical amount of 0.3 g. Thus, the design has demonstrated its ability to achieve relatively lower forward flow resistance while having good backward leakage resistance through these two experimental studies.

### Representative In-Vivo Evaluation

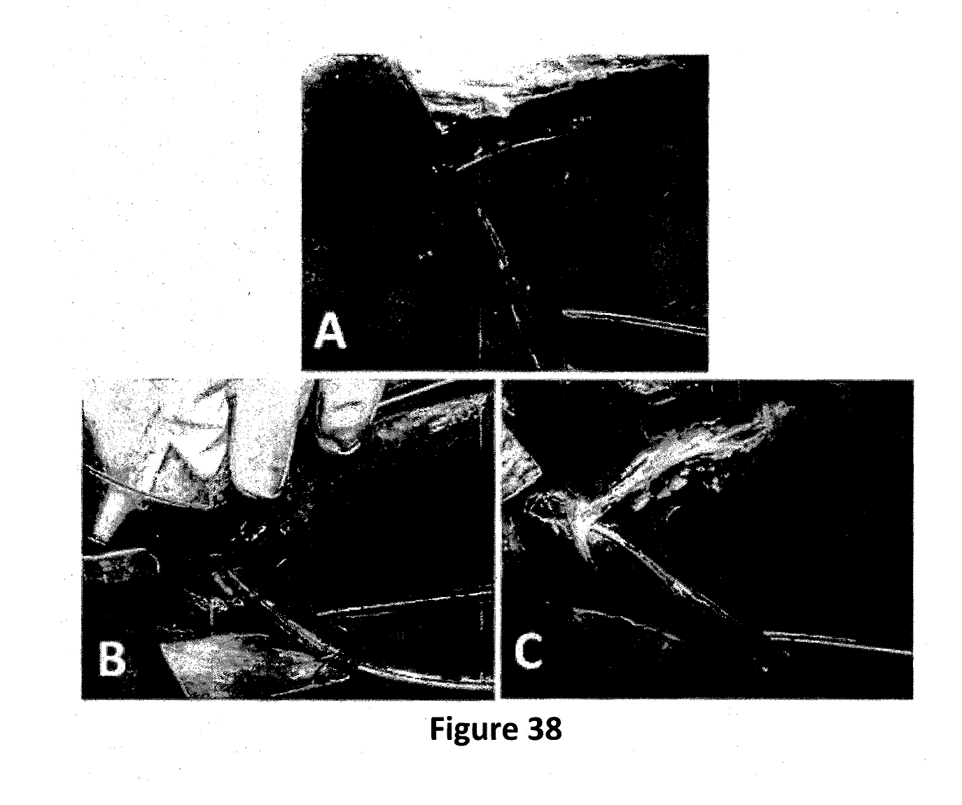

In-vivo studies on porcine models were conducted to study the ease of implantation and effectiveness of the patient specific nanocomposite voice prosthesis. The experiments were performed according to an approved Institutional Animal Care and Use Committee (IACUC) protocol. Briefly, five healthy female pigs weighing around 60 kg were anaesthetized and placed in a supine position on the operating table. A longitudinal midline neck incision was made using a mono-polar diathermy blade and the skin and muscles were laterally retracted to expose the trachea. Following which, a rectangular piece was longitudinally incised from the top portion of the trachea to expose the inner lumen. At this point, the endotracheal tube was immediately retracted from the mouth and reinserted into the end of the trachea directly to intubate the pig (FIG. 38). A puncture through the tracheao-esophageal wall was made using our previously developed Measurement And Insertion Device (MAID), and the thickness of the wall measured. The measurement allowed for the sizing of the voice prosthesis required and the latter was carefully inserted into the fistula using the MAID. Once in position, careful inspection around the prosthesis-tissue interface was carried out to ensure a good fit.

The implantations of the carbon nanocomposite voice prosthesis were successful in all five live porcine models. Closer examination of the voice prosthesis-tissue interface showed that the device was secured tightly and no gaps were visible around the deployed prosthesis. Water was flushed down the oesophagus to check for any leakages, to which there was none. The voice prosthesis will be tested in human trials in future to further assess the quality of use in patients.

### Representative Evaluation of Voice Prosthesis

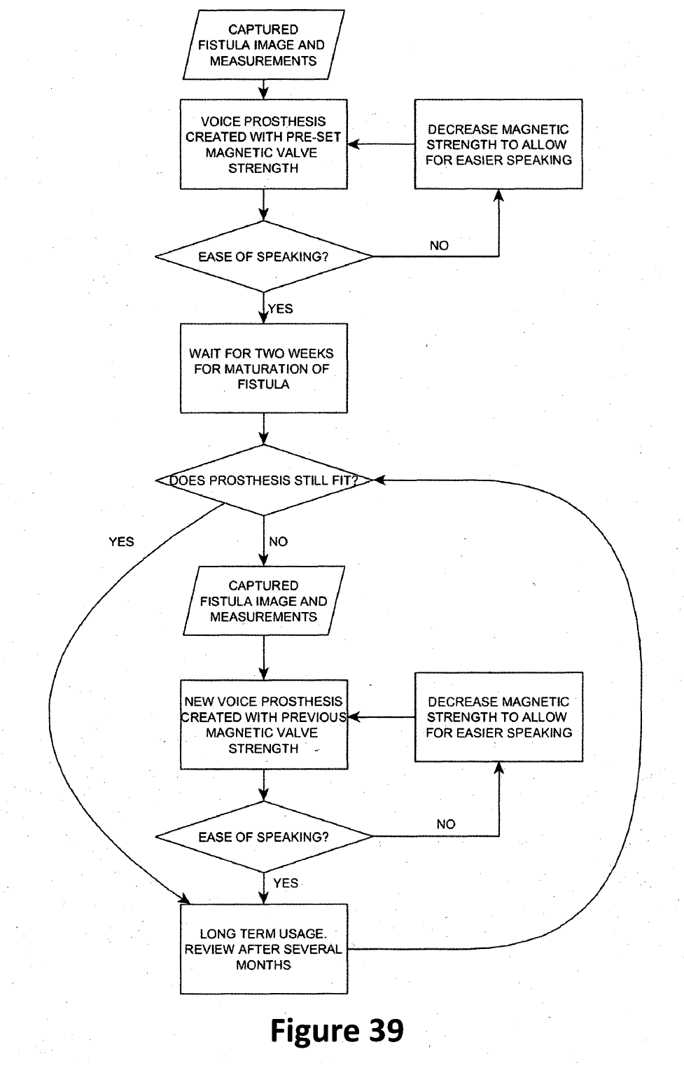

This section describes the details for the evaluation of the voice prosthesis to optimize its comfort, ease of use and dimensions. An evaluation procedure can be found in FIG. 39. The flow of the procedure starts off at the point of puncture. The shape and dimensions of the fistula are first captured via camera and the initial patient specific prosthesis is first manufactured for a snug fit into the fistula. Initially, a pre-set magnetic strength of the ball bearing is used. A ball bearing of a given magnetic strength can be selected or replaced in order to aid patient speech. The patient's ease of speech is then evaluated by a trained speech therapist and if the former encounter difficulty in speech due to poor air flow, a magnetic ball bearing of lesser strength is used for easier opening and closing of the valve. If the ease of speech is satisfactory, the patient proceeds to use this prosthesis for the next two weeks before returning back to the clinician. The second visit after 2 weeks is to assess the fit of the prosthesis in the fistula after maturation has occurred. The clinician will request the patient to drink water and will look out for any signs of leakage. If the prosthesis is deemed of adequate fit, the patient is allowed to carry on with it. If the prosthesis size is not suitable, another new prosthesis is fabricated based on the new shape and size of the fistula. Again, the ease of speaking is evaluated and the necessary adjustment is made to the magnetic strength of the ball bearing. After which, the patient is allowed to use this prosthesis for a longer term until his next review checkup.

Voice prosthesis embodiments in accordance with the present disclosure can solve the issue of irregularity by incorporating a patient specific design, shape, or geometry for a closer and better fit in a target patient's fistula. Image processing modalities and CAD design software are involved in the creation of a patient specific device. In addition, voice prosthesis embodiments in accordance with the present disclosure include a novel magnetic sealing mechanism, such as ball bearing one way valve, that can help to slow down candida formation and prevent transprosthesis leakage by ensuring better closure. Lastly, a bio-composite material including PDMS with carbon nanotubes confers better mechanical properties to the voice prosthesis. In view of the foregoing, voice prosthesis embodiments in accordance with the present disclosure can overcome limitations of existing voice prostheses.

Aspects of particular embodiments of the present disclosure address at least one aspect, problem, limitation, and/or disadvantage associated with exiting tracheal and/or voice prostheses. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, devices, structures, components, processes, or alternatives thereof, may be desirably combined into other different systems, devices, structures, components, processes, and/or applications. In addition, various modifications, alterations, and/or improvements may be made to various embodiments that are disclosed by a person of ordinary skill in the art within the scope of the present disclosure.

1.-23. (canceled)

24. A voice prosthesis comprising:
a body having a length along a body axis;
a first surface coupled to the body transverse or perpendicular to the body axis, the first surface having a first aperture disposed therein, the first surface defining a first end of the voice prosthesis;
a second surface coupled to the body transverse or perpendicular to the body axis, the second surface having a second aperture disposed therein, the second surface defining a second end of the voice prosthesis;
a passage disposed within the body along at least a portion of the body length between the first end and the second end of the voice prosthesis, the passage fluidically coupled to the first aperture and the second aperture; and
a magnetic sealing mechanism carryable by the body and configured for selectively (a) sealing the passage to prevent airflow through the passage in a direction toward the first aperture in the absence of sufficient air pressure at the first aperture, and (b) opening the passage to enable airflow through the passage in a direction toward the second aperture in the presence of sufficient air pressure at the first aperture, the magnetic sealing mechanism comprising a ball.

25. The voice prosthesis of claim 24, wherein the magnetic sealing mechanism further comprises:
a retaining link coupled to each of the ball and an inner surface of the passage; and
a magnetic seating structure carried by the second aperture, wherein the magnetic seating structure is configured to shape match a portion of an exterior surface of the ball.

26. The voice prosthesis of claim 24, wherein the magnetic sealing mechanism further comprises a magnet or a magnetic material disposed around and/or proximate to the first aperture.

27. The voice prosthesis of claim 24, wherein the body comprises at least one biocompatible polymer.

28. The voice prosthesis of claim 24, wherein the body comprises one of polydimethylsiloxane (PDMS) and polyvinyl chloride (PVC).

29. The voice prosthesis of claim 24, wherein the body comprises polydimethylsiloxane (PDMS) carrying at least one nanomaterial.

30. The voice prosthesis of claim 29, wherein the at least one nanomaterial comprises carbon nanotubes (CNTs).

31. The voice prosthesis of claim 24, wherein the body comprises
a core structure having an exterior surface; and
a chamber in which the ball resides, wherein the chamber is fluidically coupled to the passage and the second aperture.

32. The voice prosthesis of claim 31, further comprising a skin layer disposed around the exterior surface of the core structure, wherein the skin layer forms at least a portion of the second surface of the voice prosthesis.

33. The voice prosthesis of claim 32, wherein the skin layer comprises at least one biocompatible polymer carrying at least one nanomaterial.

34. The voice prosthesis of claim 33, wherein the skin layer comprises polydimethylsiloxane (PDMS) carrying carbon nanotubes (CNTs).

35. The voice prosthesis of claim 32, wherein the skin layer includes at least one cavity formed therein, in which a deformable material can be disposed.

36. The voice prosthesis of claim 24, wherein the body has a patient specific shape determined in accordance with a set of images of a fistula of a target patient.

37. The voice prosthesis of claim 32, wherein the skin layer has a patient specific shape determined in accordance with a set of images of a fistula of a target patient.

38. The voice prosthesis of claim 36, wherein the set of images includes at least one image generated by way of computed tomography (CT) and a magnetic resonance imaging (MRI).

39. A method for producing a voice prosthesis, the method comprising:
providing a body having a length along a body axis and a passage disposed along at least a portion of the body length;
providing a first surface coupled to the body transverse or perpendicular to the body axis, the first surface having a first aperture disposed therein, the first surface defining a first end of the voice prosthesis;
providing a second surface coupled to the body transverse or perpendicular to the body axis, the second surface having a second aperture disposed therein, the second surface defining a second end of the voice prosthesis,
wherein the passage is fluidically coupled to the first aperture and the second aperture; and
interfacing a magnetic sealing mechanism with the body, the magnetic sealing mechanism comprising a ball configured for selectively (a) sealing the passage to prevent airflow through the passage in a direction toward the first aperture in the absence of sufficient air pressure at the first aperture, and (b) opening the passage to enable airflow through the passage in a direction toward the second aperture in the presence of sufficient air pressure at the first aperture.

40. The method of claim 39, further comprising:
capturing a set of images of a fistula of a target patient; and
analyzing the set of captured images to determine a set of fistula parameters that define a fistula shape,
wherein providing the body comprises forming the body to have an exterior surface that exhibits a geometry or shape determined in accordance with the determined fistula shape.

41. The method of claim 39, further comprising:
capturing a set of images of a fistula of a target patient; and
analyzing the set of captured images to determine a set of fistula parameters that define a fistula shape,
wherein providing the body comprises providing a core structure carrying the passage and having a chamber configured for carrying the ball, wherein the chamber is fluidically coupled to the passage and the second aperture, and
wherein the method further comprises providing a skin layer configured for covering the core structure, wherein when the skin layer covers the core structure, the skin layer has an exterior surface that exhibits a shape determined in accordance with the determined fistula shape.

42. The method of claim 41, wherein when the skin layer covers the core structure, a portion of the skin layer forms at least a portion of the second surface of the voice prosthesis.

43. The method of claim 41, wherein providing the skin layer comprises forming the skin layer to include a cavity therein in which a deformable material is disposable.

44. The method of claim 41, wherein providing the skin layer comprises forming the skin layer by way of rapid prototyping.

45. The method of claim 40, further comprising:
generating a 3D virtual voice prosthesis model that numerically represents the voice prosthesis in accordance with the set of fistula parameters; and
simulating performance of the voice prosthesis by computationally processing the 3D virtual voice prosthesis model to generate at least one of voice prosthesis stress characteristics and voice prosthesis airflow characteristics.
